(19) United States
(12) Patent Application Publication
(10) Pub. No.: US 2026/0152721 A1
(43) Pub. Date: Jun. 4, 2026

(54) METHOD FOR GENERATING NK CELLS

(71) Applicants: APHP (ASSISTANCE PUBLIQUE - HÔPITAUX DE PARIS), Paris (FR); INSERM (INSTITUT NATIONAL DE LA SANTÉ ET DE LA RECHERCHE MÉDICALE), Paris (FR); FONDATION IMAGINE - INSTITUT DES MALADIES GÉNÉTIQUES, Paris (FR); UNIVERSITÉ PARIS CITÉ, Paris (FR)

(72) Inventors: Isabelle ANDRE, Paris (FR); Ranjita DEVI MOIRANGTHEM, Paris (FR); Tayebeh-Shabi SOHEILI, Paris (FR)

(73) Assignees: APHP (ASSISTANCE PUBLIQUE - HÔPITAUX DE PARIS), Paris (FR); INSERM (INSTITUT NATIONAL DE LA SANTÉ ET DE LA RECHERCHE MÉDICALE), Paris (FR); FONDATION IMAGINE - INSTITUT DES MALADIES GÉNÉTIQUES, Paris (FR); UNIVERSITÉ PARIS CITÉ, Paris (FR)

(21) Appl. No.: 18/706,800

(22) PCT Filed: Nov. 4, 2022
(86) PCT No.: PCT/EP2022/080831

§ 371 Date: May 2, 2024

(30) Foreign Application Priority Data
Nov. 4, 2021 (EP) 21306548.5
Jul. 29, 2022 (EP) 22306137.5

(51) Int. Cl. C12N 5/0783; A61K 35/17; A61K 40/15; A61K 40/31; A61K 40/42; C07K 14/705; C12N 15/85

(52) U.S. Cl. CPC C12N 5/0646; A61K 35/17; A61K 40/15; A61K 40/31; A61K 40/42; C07K 14/70503; C12N 15/85; C12N 2501/125; C12N 2501/2302; C12N 2501/2307; C12N 2501/2315; C12N 2501/25; C12N 2501/42; C12N 2506/11

(57) ABSTRACT

A novel method for obtaining NK cells, which includes culturing CD34+ cells in the presence of TNF-α or of a fragment thereof and of a Notch ligand or fragment thereof, thereby obtaining a first population of cells, and culturing the population of cells obtained in a cytokine-containing medium. Also, a NK cell population, and the use thereof for treating diseases, including cancer.

### FIELD OF INVENTION

The present invention relates to a method for generating CD3−CD56+ NK cells, to a NK cell population and to the use thereof as a medicament, in particular for increasing the number of NK cells in a subject in need thereof and for treating cancer and infectious diseases.

### BACKGROUND OF INVENTION

NK cells are immune innate cells that are cytotoxic and play the role of killing tumor or infected cells. NK cells are produced in the bone marrow from hematopoietic stem cells. NK cell precursors are CD3−CD161+CD56− and differentiate into immature NK cells expressing CD3−CD161+CD56+. The maturation of immature NK cells mainly takes place in the bone marrow but immature NK cells can also exit the bone marrow to mature into secondary lymphoid organs. During maturation, NK cells acquire a specific profile of membrane receptor expression, said expression profile being associated with functional NK cells.

NK cells recognize tumor and infected cells via the receptors expressed at their membrane (i.e., activating and inhibitory receptors). Inhibitory receptors recognize the major histocompatibility complex (MHC) I molecules, expressed by all the cells of the body (i.e., self-cells). Activating receptors are able to recognize non-self-molecules expressed either by self or non-self-cells (e.g., tumor or infected cells). Depending on the signals received via inhibitory and/or activating receptors, NK cells may then release the components of intracellular granules (comprising e.g., perforin and granzyme) and/or express TNF receptor ligand Fas ligand (FasL), TNF and TRAIL which binds to their corresponding receptor on target cells and/or produce proinflammatory cytokines (e.g., TNF-α and IFNγ) and then lysate the target cells (including for example cancer and infected cells).

T lymphocytes expressing a chimeric antigenic receptor are today a promising therapeutic means for targeting and killing tumor cells. However, massive secondary effects may be associated with these T cell-based immunotherapies, including, for example, cytokine storm and Graft versus Host Disease (GVHD). Because of these drawbacks, new therapeutic paths need to be investigated. NK cells, that do not trigger cytokine storm and GVHD, are an interesting alternative to T cells.

In vitro methods for obtaining NK cells were described in the prior art. However, the yield and purity of obtained NK cells is usually low, thus necessitating a sorting step of the cells prior to in vivo injection. Furthermore, these methods usually require about one month to generate NK cells. In addition, the NK cells usually poorly express activating receptors, are senescent and difficult to modify genetically.

There is thus a need to develop new methods to generate high quality NK cells in a short time period. In the present invention, the Applicants provide an in vitro method for generating CD3−CD56+ NK cells, allowing to obtain functional cytotoxic NK cells expressing activator receptors and lacking expression of some inhibitory receptors in a short time period of about two weeks.

### SUMMARY

The present invention relates to an in vitro method for generating NK cells, comprising the steps of
a) culturing CD34+ cells in presence of TNF-α or of a fragment thereof and of a Notch ligand or fragment thereof, thereby obtaining a first population of cells, and
b) culturing the population of cells obtained in step a) in a cytokine comprising medium.

In one embodiment, at step a), the cells are cultured in presence of TNF-α or of a fragment thereof and of a Notch ligand or fragment thereof for more than 5 days and less than 9 days, preferably for about 7 days.

In one embodiment, at step a), the Notch ligand is the Delta-like-4 ligand or a fragment thereof, preferably the soluble domain of the Delta-like-4 ligand.

In one embodiment, at step a), the cells are also exposed to a fibronectin fragment comprising the RGDS, connecting segment 1 (CS-1) and/or heparin-binding domain, preferably wherein the fibronectin fragment is CH-296.

In one embodiment, said CD34+ cells have been isolated from an adult donor or from cord blood cells.

In one embodiment, the cytokine comprising medium of step (b) contains at least three, preferably five cytokines, selected from the group consisting of interleukin-7 (IL-7), Stem Cell Factor (SCF), Interleukin-15 (IL-15), Interleukin-2 (IL-2) and Flt3 ligand (FLT3L).

In one embodiment, at step b), the cells are cultured in the cytokine comprising medium for more than 7 days and less than 21 days.

In one embodiment, the in vitro method comprises an additional step of transducing cells with a vector, preferably during or before step (a).

In one embodiment, the vector encodes a Chimeric Antigen Receptor (CAR).

Another object of the invention is a NK cell population susceptible to be obtained by the in vitro method of the invention, wherein more than 60% of the cells are CD3-CD56+.

In one embodiment, the cells of the NK cell population are CD3−CD56+ cells and do not express at least one inhibitory receptor selected from the group comprising KIR3DL1/DL2, KIR2DL2/DL3 and KLRG1.

In one embodiment, the cells of the NK cell population are CD3−CD56+ cells, and express at least one molecule selected from CD161 and activating receptors selected from the group comprising, NKp30, NKp44, NKp46, DNAM-1 and NKG2D. In one embodiment, the cells of the NK cell population are CD3−CD56+ cells, and express CD161, NKp30, NKp44, NKp46, DNAM-1 and NKG2D.

In one embodiment, the cells of the NK cell population are CD3−CD56+ cells, and express high levels of at least one molecule selected from CD161 and activating receptors selected from the group comprising or consisting of NKp30, NKp44, NKp46, DNAM-1 and NKG2D.

In one embodiment, the cells of the NK cell population are CD3−CD56+ cells, wherein at least 85% of said CD3−CD56+ cells express CD161 and at least one activating receptor selected from the group comprising, NKp30, NKp44, NKp46, DNAM-1 and NKG2D.

The present invention further relates to a NK cell population expressing CD161, NKp30, NKp44, NKp46, DNAM-1 and NKG2D and not expressing KIR3DL1/DL2, KIR3DL2/DL3, KLRG1.

Another object of the invention is a NK cell population as described herein for use as a medicament. Another object of the invention is a NK cell population as described herein for increasing the number of NK cells in a subject in need thereof.

Another object of the invention is a NK cell population as described herein for treating cancer, persistent viral infections and parasitic diseases.

In one embodiment, the cancer is selected from the group comprising but not limited to leukemia (i.e., acute myeloid leukemia), lymphoma (e.g., B lymphomas), non-hodgkin lymphoma, multiple myeloma, breast cancer, bladder cancer, prostate cancer, pancreatic cancer, thyroid cancer, melanoma, uterine cancer, kidney cancer, sarcoma, carcinoma, non-small cell lung cancer, oral and oropharyngeal cancer, methylcholanthrene-induced sarcomas and colorectal cancer.

In one embodiment, the cancer is selected from the group comprising but not limited to leukemia (e.g., acute myeloid leukemia, B cell acute lymphoblastic leukemia (B-ALL), T cell acute lymphoblastic leukemia (T-ALL), chronic lymphocytic leukemia (CLL), chronic myeloid leukemia), lymphoma (e.g., B lymphomas, peripheral T cell lymphoma), non-Hodgkin lymphoma, glioblastoma, neuroblastoma, multiple myeloma, cervical cancer, breast cancer (e.g., Triple-negative breast cancer), ovarian cancer, bladder cancer, prostate cancer, pancreatic cancer, gastric cancer, thyroid cancer, melanoma, uterine cancer, kidney cancer, liver cancer (e.g., hepatocellular cancer), sarcoma, carcinoma (e.g., renal cell carcinoma, breast carcinoma), small cell lung cancer, non-small cell lung cancer, pediatric solid tumor, CD133+ cancer stem cells, NKGDL+ cancer cells, PD-L1+ cancer cells, oral and oropharyngeal cancer (e.g., tongue cancer, esophageal cancer, laryngeal cancer, pharyngeal cancer) methylcholanthrene-induced sarcomas and colorectal cancer.

In one embodiment, the viral infection is selected from the group comprising but not limited to human immunodeficiency virus (HIV), herpesvirus (e.g., herpes simplex virus-1, cytomegalovirus (CMV)) influenza, retroviruses, human papillomavirus (HPV), enteroviruses (e.g., coxsackie B3 virus).

In one embodiment, the parasitic disease is selected from the group comprising but not limited to toxoplasmosis, trypanosomiasis, leishmaniasis and malaria.

### Definitions

In the present invention, the following terms have the following meanings:

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclose method.

The term “NK cell” or “natural killer cell” refers to a cytotoxic lymphocyte playing an important role in the innate immunity. NK cells are constantly in contact with other cells. NK cells express activating and inhibitory receptors at their cell surface. This mechanism allows NK cells to recognize if a cell is to be eliminated (such as, for example, a tumor cell or an infected cell) or not. In one embodiment, NK cells may be defined as “immature” (immature NK cells are generally defined by the absence of expression of CD16 and of KIR receptors), or “mature” (mature NK cells are generally defined by the expression of CD16 and of KIR receptors). Mature NK cells include memory-like NK cells.

The term “pharmaceutically acceptable excipient” (that may also be referred to as “pharmaceutically acceptable carrier”) refers to an excipient that does not produce an adverse, allergic or other untoward reaction when administered to a mammal, preferably a human. It includes any and all solvents, dispersion media, coatings, antibacterial and antifungal agents, isotonic and absorption delaying agents and the like. A pharmaceutically acceptable carrier or excipient refers to a non-toxic solid, semi-solid or liquid filler, diluent, encapsulating material or formulation auxiliary of any type. For human administration, preparations should meet sterility, pyrogenicity, general safety and purity standards as required by the regulatory offices such as the FDA or EMA.

The terms “expressing”, “positive”, or “+” and “not expressing”, “negative”, or “−” are well known in the art and refer to the expression level of a cell marker of interest, in that the expression level of the cell marker corresponding to “+” is high or intermediate or low (i.e., the cell marker is expressed or present at the cell surface), and the expression level of the cell marker corresponding to “−” is null (i.e., the cell marker is not expressed, or is absent, at the cell surface).

The term “subject” is intended to include living organisms in which an immune response can be elicited (e.g., mammals, in particular human, primates, dogs, cats, horses, sheep and the like). In one embodiment, the subject is a human. In one embodiment, a subject may be a “patient”, i.e., a warm-blooded animal, preferably a human, who/which is awaiting the receipt of, or is receiving medical care or was/is/will be the object of a medical procedure or is monitored for the development of the targeted disease or condition, such as, for example, cancer, or an infectious disease (e.g., a persistent viral infection or a parasitic disease). In one embodiment, the subject is an adult (for example a subject above the age of 18). In another embodiment, the subject is a child (for example a subject below the age of 18). In one embodiment, the subject is a male. In another embodiment, the subject is a female. In one embodiment, the subject is affected, preferably is diagnosed, with the targeted disease or condition, such as, for example, cancer, an infectious disease (e.g., a persistent viral infection or a parasitic disease). In one embodiment, the subject is at risk of developing the targeted disease or condition, such as, for example, cancer, an infectious disease (e.g., a persistent viral infection or a parasitic disease). Examples of risks factor include, but are not limited to, genetic predisposition, or familial history of the targeted disease or condition.

The terms “therapeutically effective amount” refers to an amount of the cells or of the composition as described herein, effective to achieve a particular biological result. Thus, the terms “therapeutically effective amount” mean a level or amount of a composition or a number of cells that is aimed at, without causing significant negative or adverse side effects to the target, (1) delaying or preventing the onset of the targeted disease or condition; (2) slowing down or stopping the progression, aggravation, or deterioration of one or more symptoms of the targeted disease or condition; (3) bringing about ameliorations of the symptoms of the targeted disease or condition; (4) reducing the severity or incidence of the targeted disease or condition; or (5) curing the targeted disease or condition. A therapeutically effective amount may be administered prior to the onset of the targeted disease or condition, for a prophylactic or preventive action. Alternatively, or additionally, the therapeutically effective amount may be administered after initiation of the targeted disease or condition, for a therapeutic action.

The term “Transfection” or “transduction” refers to a process by which an exogenous nucleic acid is transferred or introduced into the host cell. A “transfected” or “transduced” cell is one which has been transfected, transformed or transduced with exogenous nucleic acid, and includes the primary subject cell and its progeny.

The term “treatment” or “treating” refers to both therapeutic treatment and prophylactic or preventative measures; wherein the object is to prevent or slow down (lessen) the targeted disease or condition. Those in need of treatment include those already with the condition as well as those prone to have the condition or those in whom the condition is to be prevented. A subject is successfully “treated” for a disease or condition if, after receiving a therapeutic amount of cells or compositions as described herein, the subject shows observable and/or measurable improvement in one or more of the following: reduction in the number of pathogenic cells; reduction in the percent of total cells that are pathogenic; relief to some extent of one or more of the symptoms associated with the specific condition; reduced morbidity and mortality, and/or improvement in quality of life issues. The above parameters for assessing successful treatment and improvement in the condition are readily measurable by routine procedures familiar to a physician.

### DETAILED DESCRIPTION

The Applicants previously observed that culturing CD34+ cells in the presence of TNF-α and a Notch ligand allows to generate CD34−CD7+ progenitor T cells after 7 days of culture (WO2018/146297). However, as shown in FIG. 8, these cells strongly express transcriptional factors that are known in the art as uniquely switched on since T-cell commitment in the lymphopoiesis process, namely GATA3 and BCL11B. In view of this expression pattern, no NK cell development of these cells may be a priori expected. However, in the present invention, the Applicants surprisingly provided a method for in vitro production of NK cells, wherein, in a first step, CD34+ cells are cultured in the presence of TNF-α and of a Notch ligand or a fragment thereof.

A first object of the present invention is thus an in vitro method for generating NK cells, comprising the steps of
a) culturing CD34+ cells in presence of TNF-α or a fragment thereof and of a Notch ligand or fragment thereof, thereby obtaining a first population of cells, and
b) culturing the first population of cells in a cytokine comprising medium.

In one embodiment, CD34+ cells are isolated from cord blood cells.

In one embodiment, CD34+ are recovered from an adult donor. In one embodiment, CD34+ cells are obtained from a bone marrow puncture of from peripheral blood from adult donors. As the number of CD34+ cells is low in an adult peripheral blood (i.e., 0.15% of the cells) CD34+ cells may be mobilized from the bone marrow to the periphery (e.g., the blood) to increase the number of CD34+ cells in the peripheral blood. In one embodiment, to mobilize CD34+ cells, adult donors may be treated with granulocyte colony-stimulating factor (G-CSF) and/or plerixafor, preferably with G-CSF. Other examples of mobilizing agents include, but are not limited to, agonists of CXCR2 (e.g., MGTA 145), and analogs of plerixafor.

In one embodiment, the CD34+ cells are isolated from mobilized peripheral blood.

Methods for isolating CD34+ cells are well known in the art and include, without limitation, methods using beads coated with an antibody recognizing CD34. In one embodiment, CD34+ cells are isolated using the indirect CD34 microbead kit (Miltenyi).

In one embodiment, the CD34+ cell population used in the method of the present invention is pure at least about 90, 91, 92, 93, 94, 95, 96, 97, 98 or 99%. In one embodiment, CD34+ cells do not express markers of mature cells, preferably CD34+ cells do not express at least one marker selected from the group comprising or consisting of CD3, CD56, CD14/15, CD11b, more preferably the CD34+ cells do not express all of these markers.

In one embodiment, CD34+ cells are derived from iPSC (induced pluripotent stem cells). Examples of methods for deriving CD34+ cells from iPSC are known by the skilled artisan, and are described, for example, by John F. Tisdale in 2020 (Hematopoietic stem cells from pluripotent stem cells: Clinical potential, challenges, and future perspectives, Stem Cells Translational Medicine, Volume 9, Issue 12, December 2020, Pages 1549-1557) and by Rao and colleagues (Hematopoietic Cells from Pluripotent Stem Cells: Hope and Promise for the Treatment of Inherited Blood Disorders. Cells 2022, 11, 557).

In one embodiment, the CD34+ cells are seeded at a concentration ranging from about 10⁶ to about 10⁷ cells/mL of culture medium.

In one embodiment, the CD34+ cells are seeded at a concentration ranging from about 5 000 to about 30 000 cells/cm² of culture medium, preferably at a concentration ranging from about 10 000 to about 30 000 cells/cm².

In one embodiment, at the beginning of step (a), the CD34+ cells are seeded at a concentration ranging from about 10⁶ to about 10⁷ cells/mL of culture medium.

In one embodiment, at the beginning of step (a), the CD34+ cells are seeded at a concentration ranging from about 5 000 to about 30 000 cells/cm² of culture medium, preferably at a concentration ranging from about 10 000 to about 30 000 cells/cm².

In one embodiment, the culture vessel is selected conventional culture vessels comprising (but not limited to) culture plates from 6 to 96 wells, petri dishes, flasks, stirrer bottles, micro titer plates, test tubes, hollow fiber devices, cell foam and bags. The quantity of cells seeded may be adapted by one skilled in the art, according to the culture vessel used.

In one embodiment, the culture media used at step (a) and at step (b) are different.

In one embodiment, the culture medium used in the step a) of the method of the invention is adapted for the culture of CD34+ cells. Examples of culture medium adapted for culture of CD34+ cells include, but are not limited to, α-MEM, DMEM, RPMI 1640, IMDM, BME, McCoy's 5A, SFII (StemCell Technologies) media, Fischer's medium and X-VIVO™ medium (Lonza, Basel, Switzerland). In one embodiment, CD34+ cells are cultured in α-MEM medium (Thermo Fischer, MA, USA).

In one embodiment, the culture medium used in the present invention (in particular the culture medium of step (a) and/or the culture medium of step (b)) is feeder cell-free. In particular, in one embodiment of the present invention, the culture medium used in the present invention (in particular the culture medium of step (a) and/or the culture medium of step (b)) is free of OP-9 feeder cells.

In one embodiment, the culture medium used in the present invention (in particular the culture medium of step (a) and/or the culture medium of step (b)) is serum-free. In one embodiment, the culture medium used in the present invention (in particular the culture medium of step (a) and/or the culture medium of step (b)) is supplemented with at least about 1, 2, 3, 4, 5, 6, 7, 8, 9, 10 11, 12, 13, 14, 15, 16, 17, 18, 19 or 20% v/v of fetal bovine serum (FBS) or fetal calf serum (FCS).

In one embodiment, TNF-α is human TNF-α, having for example the sequence of SEQ ID NO: 1 (Uniprot accession number: P01375).

SEQ ID NO: 1
MSTESMIRDVELAEEALPKKTGGPQGSRRCLFLSLFSFLIVAGATTLFC
LLHFGVIGPQREEFPRDLSLISPLAQAVRSSSRTPSDKPVAHVVANPQA
EGQLQWLNRRANALLANGVELRDNQLVVPSEGLYLIYSQVLFKGQGCPS
THVLLTHTISRIAVSYQTKVNLLSAIKSPCQRETPEGAEAKPWYEPIYL
GGVFQLEKGDRLSAEINRPDYLDFAESGQVYFGIIAL

TNF-α is primarily produced as a type II transmembrane protein arranged in stable homodimers, each monomer comprising 233 amino acids in human. In human, the soluble part of human TNF-α is composed of amino acid 77 to 233 of SEQ ID NO: 1.

In one embodiment, the first culture medium (i.e., the culture medium of step (a)) comprises full-length TNF-α or a soluble fragment thereof, wherein said soluble fragment thereof may comprise or consist of amino acids 77 to 233 of SEQ ID NO: 1.

In one embodiment, TNF-α or the fragment thereof is added at day 0 of culture. In one embodiment, TNF-α or the fragment thereof is present in the culture medium since day 0 and during at least about 1, 2, 3, 4, 5, 6 or 7 days. In one embodiment, TNF-α or the fragment thereof is present in the culture medium from day 0 of step (a) to the end of step (a).

In one embodiment, TNF-α or the fragment thereof is used at a concentration ranging from about 1 to about 300 ng/mL, such as, for example, of at least about 1, 5, 10, 20, 30, 40, 50, 100, 200 or 300 ng/mL. In one embodiment, TNF-α or the fragment thereof is used at a concentration of about 10 ng/mL. However, other concentrations such as about 5, 10, 20 or 50 ng/mL are also suitable.

Notch proteins are transmembrane receptors that regulate the cellular response to a large number of environmental signals. In mammals, four Notch receptors (Notch 1-4) and five ligands (Delta-like-1, Delta-like-3, Delta-like-4, Jagged-1 and Jagged-2) have been described (Weinmaster Curr Opin Genet Dev 2000:10: 363-369).

In one embodiment, the culture medium comprises Delta-like-4, preferably human Delta-like-4 (also known as DL-4, Uniprot accession number: Q9NR61, SEQ ID NO: 2), or a fragment thereof.

SEQ ID NO: 2
MAAASRSASGWALLLLVALWQQRAAGSGVFQLQLQEFINERGVLASGRP
CEPGCRTFFRVCLKHFQAVVSPGPCTFGTVSTPVLGTNSFAVRDDSSGG
GRNPLQLPFNFTWPGTFSLIIEAWHAPGDDLRPEALPPDALISKIAIQG
SLAVGQNWLLDEQTSTLTRLRYSYRVICSDNYYGDNCSRLCKKRNDHFG
HYVCQPDGNLSCLPGWTGEYCQQPICLSGCHEQNGYCSKPAECLCRPGW
QGRLCNECIPHNGCRHGTCSTPWQCTCDEGWGGLFCDQDLNYCTHHSPC
KNGATCSNSGQRSYTCTCRPGYTGVDCELELSECDSNPCRNGGSCKDQE
DGYHCLCPPGYYGLHCEHSTLSCADSPCFNGGSCRERNQGANYACECPP
NFTGSNCEKKVDRCTSNPCANGGQCLNRGPSRMCRCRPGFTGTYCELHV
SDCARNPCAHGGTCHDLENGLMCTCPAGESGRRCEVRTSIDACASSPCF
NRATCYTDLSTDTFVCNCPYGFVGSRCEFPVGLPPSFPWVAVSLGVGLA
VLLVLLGMVAVAVRQLRLRRPDDGSREAMNNLSDFQKDNLIPAAQLKNT
NQKKELEVDCGLDKSNCGKQQNHTLDYNLAPGPLGRGTMPGKFPHSDKS
LGEKAPLRLHSEKPECRISAICSPRDSMYQSVCLISEERNECVIATEV

In one embodiment, the culture medium used in the present invention, preferably the culture medium used in step (a), comprises the soluble domain of at least one Notch ligand. In one embodiment, the soluble domain of a Notch ligand represents the extracellular portion of said ligand.

In one embodiment, the Notch ligand or fragment thereof (preferably the soluble domain of the notch ligand) is fused to a protein allowing the Notch ligand to be immobilized on a support.

In one embodiment, the Notch ligand or fragment thereof (preferably the soluble domain of the notch ligand) is fused to Biotin.

In one embodiment, the Notch ligand or fragment thereof (preferably the soluble domain of the notch ligand) is fused to a Fc region of an IgG protein, such as, for example, a human IgG protein. In one embodiment, the Notch ligand or fragment thereof (preferably the soluble domain of the notch ligand) is fused to a Fc region of an IgG2 protein, such as, for example, a human IgG2 protein (NCBI accession number: 4HAF_A, SEQ ID NO: 3).

SEQ ID NO: 3
VECPPCPAPPVAGPSVFLFPPKPKDTLMISRTPEVTCVVVDVSHEDPEV
QFNWYVDGVEVHNAKTKPREEQFNSTFRVVSVLTVVHQDWLNGKEYKCK
VSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQG
NVFSCSVMHEALHNHYTQKSLSLSPGK

In one embodiment, the culture medium used in the present invention, preferably the culture medium used in step (a), comprises DL-4 or a fragment thereof, preferably a fragment comprising or consisting of the soluble domain of the DL-4.

In one embodiment, the soluble domain of DL-4 comprises or consists of amino acids 1-526 of SEQ ID NO: 2. In another embodiment, the soluble domain of DL-4 comprises or consists of amino acids 1-525 of SEQ ID NO: 2. In another embodiment, the soluble domain of DL-4 comprises or consists of amino acids 1-524 of SEQ ID NO: 2.

In one embodiment, DL-4 or a soluble domain thereof is fused to the Fc receptor region of an IgG protein (such as, for example, a human IgG protein), in particular an IgG2 protein and preferably a human IgG2. An example of a protein comprising a soluble domain of DL-4 fused to the Fc receptor region of a human IgG2 protein is SEQ ID NO: 4.

SEQ ID NO: 4
MAAASRSASGWALLLLVALWQQRAAGSGVFQLQLQEFINERGVLASGRP
CEPGCRTFFRVCLKHFQAVVSPGPCTFGTVSTPVLGTNSFAVRDDSSGG
GRNPLQLPFNFTWPGTFSLIIEAWHAPGDDLRPEALPPDALISKIAIQG
SLAVGQNWLLDEQTSTLTRLRYSYRVICSDNYYGDNCSRLCKKRNDHFG
HYVCQPDGNLSCLPGWTGEYCQQPICLSGCHEQNGYCSKPAECLCRPGW
QGRLCNECIPHNGCRHGTCSTPWQCTCDEGWGGLFCDQDLNYCTHHSPC
KNGATCSNSGQRSYTCTCRPGYTGVDCELELSECDSNPCRNGGSCKDQE
DGYHCLCPPGYYGLHCEHSTLSCADSPCFNGGSCRERNQGANYACECPP
NFTGSNCEKKVDRCTSNPCANGGQCLNRGPSRMCRCRPGFTGTYCELHV
SDCARNPCAHGGTCHDLENGLMCTCPAGESGRRCEVRTSIDACASSPCF
NRATCYTDLSTDTFVCNCPYGFVGSRCEFPVGLPPSTMVRSVECPPCPA
PPVAGPSVFLFPPKPKDTLMISRTPEVTCVVVDVSHEDPEVQFNWYVDG
MEVHNAKTKPREEQFNSTFRVVSVLTVVHQDWLNGKEYKCKVSNKGLPA
PIEKTISKTKGQPREPQVYTLPPSREEMTKNQVSLTCLVKGFYPSDIAV
EWESNGQPENNYKTTPPMLDSDGSFFLYSKLTVDKSRWQQGNVFSCSVM
HEALHNHYTQKSLSLSPGK

An example of a protein comprising a soluble domain of DL-4 fused to the Fc receptor region of an IgG1 protein (such as, for example, a human IgG1 protein) is a commercially available product (Sino Biologicals) comprising the extracellular domain (Met 1-Pro 524) of human DLL4 (full-length DLL4 accession number NP_061947.1) fused to the Fc region of human IgG1 at the C-terminus.

In one embodiment, the Notch ligand or fragment thereof is immobilized to the culture vessel used for the culture (i.e., bound to a solid support), although it is possible that certain elements may be found in solution. In one embodiment, the Notch ligand or fragment thereof is immobilized on the surface, preferably on the inner surface, of the culture vessel. Without willing to be bound to any theory, the Applicants suggest that immobilization of the Notch ligand or fragment thereof may stabilize it in order to facilitate interaction with the CD34+ cells and thus to allow activation of the Notch receptor of the CD34+ cells. In another embodiment, the Notch ligand or fragment thereof is immobilized on the surface of beads, preferably microbeads or such as polymer or magnetic beads (with a diameter generally comprised between 1 and 5 μm), present in the culture medium.

The binding of the Notch ligand or fragment thereof (e.g., to beads or to the surface of a culture vessel) may or may not be covalent. The binding of the Notch ligand may be carried out non-covalently by allowing the Notch ligand or fragment thereof to be adsorbed onto the surface of the culture vessel or of beads. Methods for attaching a protein or peptide to beads or culture vessels are known in the art, and include, without limitation, fragment crystallizable (Fc) region of an immunoglobulin molecule (such as, e.g., human IgG); and biotin-streptavidin/neutravidin/avidin conjugation methods; and click chemistry conjugation methods.

A method to coat a culture vessel or beads with a Notch ligand is disclosed in WO2016/055396. In one embodiment, according to WO 2016/055396, around 75% of the Notch ligand, in particular DL-4, will adhere to the culture vessel surface or to the beads surface when 5 g/ml is used. In one embodiment, the composition used for coating a culture vessel or beads with a Notch ligand comprises a concentration of the Notch ligand higher or equal to 1.25 g/ml and preferably ranging from 2.5 and 5 g/ml.

In one embodiment, the culture medium used at step (a) further comprises cytokines.

In one embodiment, the culture medium used at step (a) comprises at least 1, 2 or 3 cytokines selected from the group comprising or consisting of SCF (stem cell factor), Flt3-L (Flt3 ligand), and IL-7. In one embodiment, the culture medium used at step (a) comprises at least 1, 2 or 3 cytokines selected from the group comprising or consisting of human SCF, human Flt3-L, and human IL-7.

In one embodiment, the culture medium used at step (a) comprises at least 1, 2, 3 or 4 cytokines selected from the group comprising or consisting of SCF (stem cell factor), Flt3-L (Flt3 ligand), TPO (thrombopoietin) and IL-7 (interleukin 7).

In one embodiment, the culture medium used at step (a) comprises at least 1, 2, 3 or 4 cytokines selected from the group comprising or consisting of hSCF (stem cell factor, e.g., corresponding to the uniprot accession number: P21583), hFlt3-L (Flt3 ligand, e.g., corresponding to the uniprot accession number: P49771), hTPO (thrombopoietin, e.g., corresponding to the uniprot accession number: P40225) and hIL-7 (human interleukin 7, e.g., corresponding to the uniprot accession number: P13232). hSCF, hFlt3-L, hTPO and hIL-7 are provided, for example, by Peprotech.

In one embodiment, the culture medium used at step (a) comprises SCF, (preferably hSCF). In one embodiment, the culture medium used at step (a) comprises Flt3-L (preferably hFlt3-L). In one embodiment, the culture medium used at step (a) comprises TPO (preferably hTPO). In one embodiment, the culture medium used at step (a) comprises IL-7 (preferably hIL-7).

In one embodiment, the culture medium used at step (a) comprises SCF (preferably hSCF) and Flt3-L (preferably hFlt3-L). In one embodiment, the culture medium used at step (a) comprises SCF (preferably hSCF) and TPO (preferably hTPO). In one embodiment, the culture medium used at step (a) comprises SCF (preferably hSCF) and IL-7 (preferably hIL-7). In one embodiment, the culture medium used at step (a) comprises Flt3-L (preferably hFlt3-L) and TPO (preferably hTPO). In one embodiment, the culture medium used at step (a) comprises Flt3-L (preferably hFlt3-L) and IL-7 (preferably hIL-7). In one embodiment, the culture medium used at step (a) comprises TPO (preferably hTPO) and IL-7 (preferably hIL-7).

In one embodiment, the culture medium comprises SCF (preferably hSCF), Flt3-L (preferably hFlt3-L) and TPO (preferably hTPO). In one embodiment, the culture medium comprises SCF (preferably hSCF), Flt3-L (preferably hFlt3-L) and IL-7 (preferably hIL-7). In one embodiment, the culture medium comprises SCF (preferably hSCF), TPO (preferably hTPO) and IL-7 (preferably hIL-7). In one embodiment, the culture medium comprises Flt3-L (preferably hFlt3-L), TPO (preferably hTPO) and IL-7 (preferably hIL-7).

In one embodiment, the culture medium comprises SCF (preferably hSCF), Flt3-L (preferably hFlt3-L), TPO (preferably hTPO) and IL-7 (preferably hIL-7).

In one embodiment, the concentration of hSCF ranges from about 2 to about 300 ng/mL, preferably from about 40 to about 200 ng/mL and more preferably is of about 100 ng/mL.

In one embodiment, the concentration of hFlt3-L ranges from about 2 to about 300 ng/mL, preferably from about 40 to about 200 ng/mL and more preferably is of about 100 ng/mL.

In one embodiment, the concentration of hTPO ranges from about 2 to about 300 ng/mL, preferably from about 40 to about 200 ng/mL and more preferably is of about 100 ng/mL.

In one embodiment, the concentration of hIL-7 ranges from about 2 to about 300 ng/mL, preferably from about 40 to about 200 ng/mL and more preferably is of about 100 ng/mL.

In one embodiment, the culture medium of step (a) does not comprise IL-3.

In one embodiment, the culture medium comprises fibronectin or a fibronectin fragment (fibronectin may have a sequence corresponding to the uniprot accession number: P02751, SEQ ID NO: 5). In one embodiment, the fibronectin fragment comprises or consists of an RGDS motif, a connecting segment 1 (CS-1) motif and/or a heparin binding domain, preferably, the fibronectin fragment comprises or consists of an RGDS motif, a CS-1 motif and a heparin binding domain.

Fibronectin is a protein, which in its natural form is a v-shaped large dimer of 100 nm long and 460 kDa. The two monomers are connected by two disulfide bridges at their C-terminus. The term “fibronectin” or “fibronectin fragment” is understood to mean the natural fibronectin protein (i.e., any isoform produced by alternative splicing), but also a monomer of this protein, or a fragment of this protein (containing, when specified, the RGDS motif, CS-1 motif and heparin binding site).

An example of a fibronectin fragment which is particularly suitable for carrying out the process herein disclosed is Retronectin®. This protein corresponds to a fragment of a human fibronectin (CH-296 fragment, Kimizuka et al., J Biochem., 1991 August 110 (2): 284-91, Chono et al., J Biochem 2001 September 130 (3): 331-4) and contains the cell-binding C domain (comprising the RGDS motif, the heparin-binding domain and the CS-1 motif). This protein is sold in particular by the companies Takara Bio Inc. (Shiga, Japan), Clinisciences (Nanterre, France, also called NovoNectin®) and Fisher scientific (Hampton, United-States).

The term “RGDS motif” is intended to designate any peptide or protein that contains the RGDS (SEQ ID NO: 6) pattern, so that it can bind integrin VLA-5. Such peptide or protein can be tested for its ability to bind VLA-5 integrin by methods known and reported in the art. RGDS motif binds to integrin VLA-5 (Very Late Antigen-5), which is a dimer composed of CD49e (alpha5) and CD29 (beta1).

Heparin-binding domains are known in the art and present in numerous proteins that bind to heparin. Their sequence is generally XBBXBX or XBBBXXBX (B=basic amino acid; X=hydropathic amino acid; Cardin and Weintraub, Arterioscler Thromb Vasc Biol. 1989; 9:21-32, SEQ ID NO: 7 and SEQ ID NO: 8). Presence of such a heparin-binding domain is particularly favorable when the CD34+ cells are exposed to a viral (especially a retroviral) vector in order to transduce them and obtain T cell progenitors expressing a transgene.

A a amino 25 acids peptide CS-1 motif is (DELPQLVTLPHPNLHGPEILDVPST, SEQ ID NO: 9), as described by Wayner et al., 1989, J. Cell Biol. 109:1321). The CS-1 motif binds to the VLA-4 (Very Late Antigen-4) receptor. VLA-4 is a dimer integrin, composed of CD49d (alpha 4) and CD29 (beta 1).

In one embodiment, the fibronectin or fibronectin fragment is immobilized (i.e., bound to a solid support). The binding of the fibronectin or fibronectin fragment (e.g., to beads or to the surface of a culture vessel) may or may not be covalent. In one embodiment, the fibronectin or fibronectin fragment is immobilized to the inner surface of the culture vessel (although it is possible that certain elements may be found in solution). In another embodiment, the fibronectin or fibronectin fragment is immobilized on the surface of beads, preferably microbeads or such as polymer or magnetic beads (with a diameter generally comprised between 1 and 5 μm). In one embodiment, the Notch ligand or fragment thereof and the fibronectin or fragment thereof are immobilized on the same beads. In another embodiment, the Notch ligand or fragment thereof and the fibronectin or fragment thereof are immobilized on the distinct beads.

In one embodiment, immobilization of the fibronectin or fibronectin fragment is carried out non-covalently by allowing the fibronectin or fragment thereof to be adsorbed onto the inner surface of the culture vessel or onto the surface of beads. Methods for attaching a protein or peptide to beads or to the surface of a culture vessel are known in the art and are listed hereinabove.

A method to coat a culture vessel or beads with fibronectin or a fragment thereof is disclosed in WO2016/055396. In one embodiment, the composition used for coating a culture vessel or beads with fibronectin or a fragment thereof comprises a concentration of fibronectin or a fragment thereof ranging from 10 and 100 μg/ml, preferably of about 25 μg/ml.

In one embodiment, the CD34+ cells are cultured in presence of TNF-α or of a fragment thereof and of a Notch ligand or of a fragment thereof during at least about 4, 5, 6, 7, 8, 9 or 10 days, preferably during at least about 5 or 6 days. In one embodiment, the CD34+ cells are cultured in presence of TNF-α or of a fragment thereof and of a Notch ligand or of a fragment thereof during about 4, 5, 6, 7, 8, 9 or 10 days, preferably during about 7 days. In one embodiment, the CD34+ cells are cultured in presence of TNF-α or of a fragment thereof and of a Notch ligand or of a fragment thereof during at most about 10 days.

In one embodiment, the population of cells obtained at step (a) may be injected in vivo to a human subject to generate mature NK cells.

In one embodiment, the cytokine comprising culture medium used for the step b) of the method of the invention is adapted for the culture of NK cell progenitors. In one embodiment, the culture medium is selected from the group comprising RPMI Glutamax medium (Thermo Fischer, MA, USA), StemSpan serum free medium (Stem Cell Technologies, Vancouver, Canada), Serum-free CellGro SCGM medium (Bioz, CA, USA), CellGro DC medium (CellGenix, Freiburg, Germany), Glycostem Basal Growth medium (Clear Cell Technologies, Beernem, Belgium) and Alpha MEM (Thermo Fischer, MA, USA) preferably the RPMI Glutamax medium (Thermo Fischer, MA, USA).

In one embodiment, the cytokine comprising culture medium used at step (b) is feeder cell-free.

In one embodiment, the cytokine comprising culture medium used at step (b) is serum-free. In one embodiment, the cytokine comprising culture medium used at step (b) is supplemented with at least about 1, 2, 3, 4, 5, 6, 7, 8, 9, 10 11, 12, 13, 14, 15, 16, 17, 18, 19 or 20%, preferably about 10% of fetal bovine serum (FBS) or fetal calf serum (FCS).

In one embodiment, at step (b), the cytokine comprising culture medium does not comprise TNF-α or fragment thereof. In one embodiment, at step (b), the cytokine comprising culture medium does not comprise a Notch ligand or fragment thereof. In one embodiment, at step (b), the cytokine comprising culture medium does not comprise TNF-α or fragment thereof nor a Notch ligand or fragment thereof.

In one embodiment, the cytokine comprising culture medium comprises IL-15, preferably hIL-15. In one embodiment, the cytokine comprising culture medium comprises SCF, preferably hSCF. In one embodiment, the cytokine comprising culture medium comprises Flt3-L, preferably hFlt3-L. In one embodiment, the cytokine comprising culture medium comprises IL-7, preferably hIL-7. In one embodiment, the cytokine comprising culture medium comprises IL-2, preferably hIL-2.

In one embodiment, the cytokine comprising culture medium comprises 1, 2, 3, 4 or 5 cytokines selected from the group comprising or consisting of IL-15, SCF, Flt3-L, IL-7, and IL-2.

In one embodiment, the cytokine comprising culture medium comprises 1, 2, 3, 4 or 5 cytokines selected from the group comprising or consisting of hIL-15 (human interleukin 15, e.g., corresponding to accession number: P40933, that may be provided by Peprotech), hSCF (stem cell factor), hFlt3-L, hIL-7, and hIL-2 (human interleukin 2, e.g., corresponding to the accession number: P60568, that may be provided, for example, by Novartis).

In one embodiment, the cytokine comprising culture medium comprises IL-15 (preferably hIL-15) and SCF (preferably hSCF). In one embodiment, the cytokine comprising culture medium comprises IL-15 (preferably hIL-15) and Flt3-L (preferably hFlt3-L). In one embodiment, the cytokine comprising culture medium comprises IL-15 (preferably hIL-15) and IL-7 (preferably hIL-7). In one embodiment, the cytokine comprising culture medium comprises IL-15 (preferably hIL-15) and IL-2 (preferably hIL-2). In one embodiment, the cytokine comprising culture medium comprises SCF (preferably hSCF) and Flt3-L (preferably hFlt3-L). In one embodiment, the cytokine comprising culture medium comprises SCF (preferably hSCF) and IL-7 (preferably hIL-7). In one embodiment, the cytokine comprising culture medium comprises SCF (preferably hSCF) and IL-2 (preferably hIL-2). In one embodiment, the cytokine comprising culture medium comprises Flt3-L (preferably hFlt3-L) and IL-7 (preferably hIL-7). In one embodiment, the cytokine comprising culture medium comprises Flt3-L (preferably hFlt3-L) and IL-2 (preferably hIL-2). In one embodiment, the cytokine comprising culture medium comprises IL-7 (preferably hIL-7) and IL-2 (preferably hIL-2).

In one embodiment, the cytokine comprising culture medium comprises IL-15 (preferably hIL-15), SCF (preferably hSCF) and Flt3-L (preferably hFlt3-L). In one embodiment, the cytokine comprising culture medium comprises IL-15 (preferably hIL-15), SCF (preferably hSCF) and IL-7 (preferably hIL-7). In one embodiment, the cytokine comprising culture medium comprises IL-15 (preferably hIL-15), SCF (preferably hSCF) and IL-2 (preferably hIL-2). In one embodiment, the cytokine comprising culture medium comprises IL-15 (preferably hIL-15), Flt3-L (preferably hFlt3-L) and IL-7 (preferably hIL-7). In one embodiment, the cytokine comprising culture medium comprises IL-15 (preferably hIL-15), Flt3-L (preferably hFlt3-L) and IL-2 (preferably hIL-2). In one embodiment, the cytokine comprising culture medium comprises IL-15 (preferably hIL-15), IL-7 (preferably hIL-7) and IL-2 (preferably hIL-2). In one embodiment, the cytokine comprising culture medium comprises SCF (preferably hSCF), Flt3-L (preferably hFlt3-L) and IL-7 (preferably hIL-7). In one embodiment, the cytokine comprising culture medium comprises SCF (preferably hSCF), Flt3-L (preferably hFlt3-L) and IL-2 (preferably hIL-2). In one embodiment, the cytokine comprising culture medium comprises SCF (preferably hSCF), IL-7 (preferably hIL-7) and hIL-2. In one embodiment, the cytokine comprising culture medium comprises Flt3-L (preferably hFlt3-L), IL-7 (preferably hIL-7) and IL-2 (preferably hIL-2).

In one embodiment, the cytokine comprising culture medium comprises IL-15 (preferably hIL-15), SCF (preferably hSCF), Flt3-L (preferably hFlt3-L) and IL-7 (preferably hIL-7). In one embodiment, the cytokine comprising culture medium comprises IL-15 (preferably hIL-15), SCF (preferably hSCF), Flt3-L (preferably hFlt3-L) and IL-2 (preferably hIL-2). In one embodiment, the cytokine comprising culture medium comprises IL-15 (preferably hIL-15), Flt3-L (preferably hFlt3-L), IL-7 (preferably hIL-7) and IL-2 (preferably hIL-2). In one embodiment, the cytokine comprising culture medium comprises IL-15 (preferably hIL-15), SCF (preferably hSCF), IL-7 (preferably hIL-7) and IL-2 (preferably hIL-2). In one embodiment, the cytokine comprising culture medium comprises SCF (preferably hSCF), Flt3-L (preferably hFlt3-L), IL-7 (preferably hIL-7) and IL-2 (preferably hIL-2).

In one embodiment, the cytokine comprising culture medium comprises IL-15, SCF (stem cell factor), Flt3-L, IL-7, and IL-2.

In one embodiment, the cytokine comprising culture medium comprises hIL-15, hSCF (stem cell factor), hFlt3-L, hIL-7, and hIL-2.

In one embodiment, the cytokine comprising culture medium does not comprise IL-12 (preferably human IL-12) and/or IL-18 (preferably human IL-18). In one embodiment, the cytokine comprising culture medium does not comprise IL-12 (preferably human IL-12) nor IL-18 (preferably human IL-18).

In one embodiment, the cytokine comprising culture medium further comprises IL-12, preferably human IL-12. In one embodiment, the cytokine comprising culture medium further comprises IL-18, preferably human IL-18. In one embodiment, the cytokine comprising culture medium further comprises IL-12 (preferably human IL-12) and IL-18 (preferably human IL-18).

In one embodiment, the cytokine comprising culture medium contains 1, 2, 3, 4, 5, 6 or 7 cytokines, selected from the group comprising or consisting of Interleukin-7 (preferably hIL-7), Stem Cell Factor (preferably hSCF), Interleukin-15 (preferably hIL-15), Interleukin-2 (preferably hIL-2), Interleukin-18 (preferably hIL-18), Interleukin-12 (preferably hIL-12) and Flt3 ligand (preferably hFLT3L). In one embodiment, the cytokine comprising culture medium comprises Interleukin-7 (preferably hIL-7), Stem Cell Factor (preferably hSCF), Interleukin-15 (preferably hIL-15), Interleukin-2 (preferably hIL-2), Interleukin-18 (preferably hIL-18), Interleukin-12 (preferably hIL-12) and Flt3 ligand (preferably hFLT3L).

In one embodiment, the concentration of the cytokine hSCF ranges from about to about 200 ng/mL, preferably from about 20 to about 50 ng/mL and more preferably is of about 50 ng/mL.

In one embodiment, the concentration of the cytokine hFlt3-L ranges from about 10 to about 200 ng/mL, preferably from about 20 to about 50 ng/mL and more preferably 10 is of about 50 ng/mL.

In one embodiment, the concentration of the cytokine hIL-7 ranges from about 10 to about 200 ng/mL, preferably from about 20 to about 50 ng/mL and more preferably is of about 20 ng/mL.

In one embodiment, the concentration of the cytokine hIL-15 ranges from about 10 to about 200 ng/mL, preferably from about 20 to about 50 ng/mL and more preferably is of about 20 ng/mL.

In one embodiment, the concentration of the cytokine hIL-2 ranges from about 200 IU/mL to about 1000 IU/mL, preferably is of about 500 IU/mL.

In one embodiment, the concentration of the cytokine hIL-12 ranges from about 0.01 ng/mL to about 100 ng/mL, preferably ranges from about 0.1 ng/mL to about 50 ng/ml or from about 0.1 ng/mL to about 10 ng/mL, more preferably is of about 10 ng/mL.

In one embodiment, the concentration of the cytokine hIL-18 ranges from about 0.1 ng/mL to about 200 ng/mL, preferably ranges from about 0.5 ng/mL to about 100 ng/mL, more preferably is of about 100 ng/mL.

In one embodiment, at step (b), the cells are cultured during at least about, or for about, 4, 5, 6, 7, 8, 9, 10, 11, 12, 13, 14, 15, 16, 17, 18, 19, 20 or 21 days, preferably during a time period ranging from about 7 to about 14 days, more preferably for about 10, 11, 12, 13, or 14 days, thereby obtaining NK cells.

In one embodiment, at the beginning of step (b), the CD34+ cells are seeded at a concentration ranging from about 10⁶ to about 10⁷ cells/mL of cytokine comprising culture medium.

In one embodiment, the method of the invention further comprises a washing step of the cells obtained at the end of step (b).

In one embodiment, the NK cells obtained at step (b) are frozen according to methods known in the art. In one embodiment, the NK cells are centrifuged (e.g., at 1500 rpm for 5 min) and resuspended in a freezing medium (e.g., comprising 90% v/v FBS and 10% v/v DMSO).

In one embodiment, the NK cells obtained at step (b) are thawed before use, for example at 37° C. in a water bath.

In one embodiment, the method of the invention may also comprise a step of conditioning the NK cells obtained at step (b) in a pouch for injection to a patient. In one embodiment, the NK cells are reconditioned in a saline solution containing 5% HSA such as Albunorm™ 5% 50 g/L (Octopharma, Lingolsheim, France).

In one embodiment, during or after the step (b) of the method of the invention, the cells may further be cultured in a culture medium adapted for the maturation of NK cells. Thus, in one embodiment, the method of the invention further comprises a step (b′) of culture of the cells during or after the step (b) in a maturation culture medium

In one embodiment, the step (b′) is performed after step (b).

In one embodiment, the step (b′) is performed during step (b) meaning that the step (b) comprises:
a step (b₁) of culture of cells in a cytokine comprising medium
a step (b′) of culture of cells in a maturation culture medium and
a step (b₂) of culture of cells in a cytokine comprising medium.

In one embodiment, at step (b′), the cells are cultured during at least about, or for during at least about 1, 2, 3, 4, 5, 6, 7, 8, 9, 10, 11, 12, 13, 14, 15, 16, 17, 18, 19, 20 hours preferably during a time period ranging from about 6 to about 12 hours, more preferably for about 6 hours in the maturation culture medium, thereby obtaining mature NK cells.

In one embodiment, the step (b′) is performed before step (b) as a pre-activated culture step during at least about, or during about 1, 2, 3, 4, 5, 6, 7, 8, 9, 10, 11, 12, 13, 14, 15, 16, 17, 18, 19, 20 hours, preferably during about 12 hours in the maturation culture medium.

In one embodiment, at step (b₁), the cells are cultured during at least about, or for about, 4, 5, 6, 7, 8 days, preferably during a time period ranging from about 4 to about 7 days, more preferably for about 7 days in the cytokine comprising medium.

In one embodiment, at step (b₂), the cells are cultured during at least about, or for about, 4, 5, 6, 7, 8 days, preferably during a time period ranging from about 4 to about 7 days, more preferably for about 7 days in the cytokine comprising medium.

In one embodiment, the maturation culture medium is selected from the group comprising RPMI (Thermo Fischer, MA, USA), StemSpan serum free medium (Stem Cell Technologies, Vancouver, Canada), Serum-free CellGro SCGM medium (Bioz, CA, USA), CellGro DC medium (CellGenix, Freiburg, Germany), Glycostem Basal Growth medium (Clear Cell Technologies, Beernem, Belgium) and Alpha MEM (Thermo Fischer, MA, USA) preferably the RPMI (Thermo Fischer, MA, USA).

In one embodiment, the maturation culture medium is feeder cell-free.

In one embodiment, the maturation culture medium is serum-free. In one embodiment, the maturation culture medium is supplemented with at least about 1, 2, 3, 4, 5, 6, 7, 8, 9, 10 11, 12, 13, 14, 15, 16, 17, 18, 19 or 20%, preferably with about 10% of fetal bovine serum (FBS) or fetal calf serum (FCS).

In one embodiment, at step (b′), the maturation culture medium does not comprise TNF-α or a fragment thereof. In one embodiment, at step (b′), the maturation culture medium does not comprise a Notch ligand or fragment thereof. In one embodiment, at step (b′), the maturation culture medium does not comprise TNF-α or a fragment thereof nor a Notch ligand or fragment thereof.

In one embodiment, the maturation culture medium comprises IL-12, IL-15 and/or IL-18.

In one embodiment, the maturation culture medium comprises hIL-12 (human interleukin-12, that may comprise a dimer of p35 and p40 proteins e.g., corresponding to the uniprot accession numbers: P29459 and P29460), hIL-15 and/or hIL-18 (human interleukin-18, e.g., corresponding to the uniprot accession number: Q14116). h-IL12 may be provided by, for example Peprotech or Miltenyi Biotech and h-IL18 may be provided by, for example, MBL International Corporation or R&D Biosystems.

In one embodiment, the cytokine comprising maturation culture medium comprises IL-12 (preferably hIL-12). In one embodiment, the cytokine comprising maturation culture medium comprises IL-15 (preferably hIL-15). In one embodiment, the cytokine comprising maturation culture medium comprises IL-18 (preferably hIL-18).

In one embodiment, the cytokine comprising maturation culture medium comprises IL-12 (preferably hIL-12) and IL-15 (preferably hIL-15). In one embodiment, the cytokine comprising maturation culture medium comprises IL-12 (preferably hIL-12) and IL-18 (preferably hIL-18). In one embodiment, the cytokine comprising maturation culture medium comprises IL-15 (preferably hIL-15) and IL-18 (preferably hIL-18).

In one embodiment, the cytokine comprising maturation culture medium comprises IL-12 (preferably hIL-12), IL-15 (preferably hIL-15) and IL-18 (preferably hIL-18).

In one embodiment, the concentration of the cytokine hIL-12 ranges from about 5 to 200 ng/mL, preferably from about 5 to 50 ng/ml and more preferably is of about 10 ng/mL.

In one embodiment, the concentration of the cytokine hIL-12 ranges from about 0.01 ng/mL to about 100 ng/mL, preferably ranges from about 0.1 ng/mL to about 50 ng/mL or from about 0.1 ng/mL to about 10 ng/mL, more preferably is of about 10 ng/mL.

In one embodiment, the concentration of the cytokine hIL-15 ranges from about 10 to 200 ng/mL, preferably from about 20 to 100 ng/mL and more preferably is of about 50 ng/mL.

In one embodiment, the concentration of the cytokine hIL-18 ranges from about to 200 ng/mL, preferably from about 20 to 100 ng/mL and more preferably is of about 50 ng/mL.

In one embodiment, the concentration of the cytokine hIL-18 ranges from about 0.1 ng/mL to about 200 ng/mL, preferably ranges from about 0.5 ng/mL to about 100 ng/mL, more preferably is of about 100 ng/mL.

In one embodiment, the maturation culture medium comprises Interleukin-7 (preferably hIL-7), Stem Cell Factor (preferably hSCF), Interleukin-15 (preferably hIL-15), Interleukin-2 (preferably hIL-2), Flt3 ligand (preferably hFLT3L), Interleukin-18 (preferably hIL-18) and Interleukin-12 (preferably hIL-12). In one embodiment, concentrations of the cytokines of the maturation culture medium are equivalent to the concentrations of cytokines present in the cytokine comprising medium, and are detailed hereinabove.

In one embodiment, the method of the invention further comprises a washing step of the cells obtained at the end of step (b′) or (b₂).

In one embodiment, the NK cells obtained at step (b′) or (b₂) are frozen according to methods known in the art. In one embodiment, the NK cells are centrifuged (e.g., at 1500 rpm for 5 min) and resuspended in a freezing medium (e.g., 90% v/v FBS and 10% v/v DMSO).

In one embodiment, the NK cells obtained at step (b′) or (b₂) are thawed before use, for example at 37° C. in a water bath.

In one embodiment, the method of the invention may also comprise a step of conditioning the NK cells (preferably the mature NK cells) obtained at step (b′) or (b₂) in a pouch for injection to a patient. In one embodiment, the NK cells are reconditioned in a saline solution containing 5% HSA such as Albunorm™ 5% 50 g/L (Octopharma, Lingolsheim, France).

In one embodiment, the method of the invention further comprises a genetic modification step. In one embodiment, the method of the invention further comprises one or more genetic modification step(s).

In one embodiment, the genetic modification step(s) correspond(s) to a gene disruption step, a gene correction step or a gene addition step, preferably a gene addition step. In one embodiment, the genetic modification step(s) is/are carried out by a method selected from the group comprising, but not limited to, transfection, transduction or gene editing.

Examples of methods of gene editing that may be used in the present invention include, but are not limited to, methods based on engineered nucleases, methods based on recombinant Adeno-Associated Virus (or AAV), methods based on Transposons (e.g., Sleeping Beauty transposon system), methods based on homologous recombination, conditional targeting using site-specific recombinases (e.g., Cre-LoxP and Flp-FRT systems), and Multiplex Automated Genomic Engineering (MAGE). Other examples of methods of gene editing that may be used in the present invention include, but are not limited to, methods based on nickases.

Non-limiting examples of engineered nucleases include, but are not limited to, clustered regularly interspaced short palindromic repeats (CRISPR) transcription-activator like effector nuclease (TALEN), zinc finger endonuclease (ZFN), meganuclease (mn, also known as homing endonuclease), or megaTAL (combining a TAL effector with a mn cleavage domain). Other non-limitative examples of engineered nucleases are base editors or prime editors.

In one embodiment, an exogenous nucleic acid sequence expressing a gene of interest is introduced into the cells, preferably before or during step (a) of the method.

In one embodiment, the transduction or transfection of CD34+ cells is carried out before step (a) of the method of the present invention.

In one embodiment, before step (a) of the method, CD34+ cells are preactivated during at least about 1, 2, 3, 4, 5, 6, 7, 8, 9, 10, 11, 12, 13, 14, 15, 16, 17, 18, 19, 20 hours, preferably overnight before being transduced. In one embodiment, before step (a) of the method, CD34+ cells are preactivated during at least about 1 day before being transduced. In one embodiment, pre-activation comprises or consists in culture of cells in a culture medium equivalent to the culture medium of step (a) but lacking TNF-α or a fragment thereof. In another embodiment, pre-activation comprises or consists in culture of cells in a culture medium equivalent to the culture medium of step (a). According to this embodiment, the duration of the combination of the gene modification step and of step a) is of about at least about 4, 5, 6, 7, 8, 9 or 10 days, preferably of at least about 5 or 6 days. In one embodiment, the duration of the combination of the gene modification step and of step a) is of about 4, 5, 6, 7, 8, 9 or 10 days, preferably of about 7 days.

In one embodiment, the transduction or transfection of CD34+ cells is carried out during step (a) of the method of the present invention.

In one embodiment, the transduction is performed in absence of DL-4 and fibronectin. In one embodiment, the transduction is performed in the presence of DL-4 and fibronectin. In one embodiment, the transduction is performed in the presence of TNF-α or of a fragment thereof. In one embodiment, the transduction is performed in the absence of TNF-α or of a fragment thereof.

In one embodiment, the transduction is performed in the culture medium of step (a). In one embodiment, the transduction is performed in presence of at least one of the cytokines selected from the group comprising or consisting of SCF (preferably hSCF), Flt3-L (preferably hFlt3-L), and IL-7 (preferably hIL-7), h-IL3, more preferably with the three cytokines. In one embodiment, the transduction is performed in presence of at least one of the cytokines selected from the group comprising or consisting of SCF (preferably hSCF), TPO (preferably hTPO), Flt3-L (preferably hFlt3-L), and IL-7 (preferably hIL-7), h-IL3, more preferably with the four cytokines. In one embodiment, the transduction is performed in presence of at least one of the cytokines selected from the group comprising hSCF, hTPO, hFlt3-L, hIL-7, h-IL3, more preferably with the five cytokines. In one embodiment, the cytokines are used at a concentration of at least 20 ng/ml to 300 ng/mL, more preferably the cytokines are used at a concentration of 20 ng/ml, 100 ng/mL or 300 ng/mL.

In one embodiment, the transduction is performed during at least about 1, 2, 3, 4, 5, 6, 7, 8, 9 or 10 hours, preferably during at least 6 hours.

In one embodiment, for the transduction step, the CD34+ cells are seeded at a concentration ranging from about 10⁶ to about 10⁷ cells/mL of culture medium.

In one embodiment, after transduction, the CD34+ cells are washed and then step (a) of the method of the invention is performed.

In one embodiment, the exogenous nucleic acid sequence to be introduced into the cells encodes a Chimeric Antigenic Receptor (CAR). A CAR is a cell surface protein that recognizes an antigen, such as, for example, a cell surface protein specifically expressed by the target cells (e.g., expressed by cancer cells or infected cells).

In one embodiment, the exogenous nucleic acid sequence to be introduced into the cells encodes a protein selected from the group comprising or consisting of cytokines or cytokines receptors or variant thereof (such as, for example, variants of cytokines or cytokines receptors with increased stability). In one embodiment, the exogenous nucleic acid sequence encodes a chimeric cytokine receptor or orthogonal cytokine-receptor pairs.

In one embodiment, the exogenous nucleic acid sequence encodes IL-15 or a variant thereof.

In one embodiment, the exogenous nucleic acid sequence to be introduced into the cells encodes CD16 or a variant thereof (such as, for example, cleavage resistant variant of CD16).

In one embodiment, the genetic modification step(s) is/are a gene disruption step, aiming at decreasing or abolishing expression of specific genes. Examples of genes that can be deleted include, but are not limited to, genes from the group comprising or consisting of PD1, TIGIT, LAG-3, TIM-3, Cytokine induced STAT inhibitor (CIS) and Signal regulatory protein α (SIRPα).

In one embodiment, the genetic modification step(s) is/are a gene disruption step, aiming at decreasing or abolishing expression of specific genes. Examples of genes that can be deleted include, but are not limited to, genes from the group comprising or consisting of PD1, TIGIT, LAG-3, TIM-3, TGFB2, Cytokine induced STAT inhibitor (CIS) and Signal regulatory protein α (SIRPα).

Another object of the present invention is a NK cell population susceptible to be obtained, or obtained, by the in vitro method of the invention.

In one embodiment, the NK cells of the invention are CD3−CD56+ NK cells. In one embodiment, the NK cell population of the invention comprises CD3−CD56+ NK cells.

In one embodiment, the CD3−CD56+ NK cells generated by the method of the invention have a purity of at least about 60, 65, 70, 75, 80, 81, 82, 83, 84, 85, 86, 87, 88, 89, 90, 91, 92, 93, 94, or 95%, i.e., more than 60% of the cells recovered at the end of step b) are CD3−CD56+ NK cells.

NK cells recognize tumor and infected cells by means of receptors expressed at their cell surface membrane, including activating and inhibitory receptors.

In one embodiment, the NK cells susceptible to be obtained, or obtained, by the method of the invention express at least one activating receptor selected from the group comprising or consisting of KIRDS1/S2, KIR2DS4, KIR2DL4, CD94/NKG2C, KIR3DL2, CD16, NKG2D, NCRs, DNAM-1, 2B4, NTBA and NKp80.

In one embodiment, the NK cells susceptible to be obtained, or obtained, by the method of the invention express at least one molecule selected from CD161 and activating receptors selected from the group comprising or consisting of NKp30, NKp44, NKp46, DNAM1 and NKG2D. In one embodiment, the NK cells susceptible to be obtained, or obtained, by the method of the invention are or comprise CD3−CD56+ cells, wherein at least 80%, preferably at least 85% (e.g., at least 80, 81, 82, 83, 84, 85, 86, 87, 88, 89, 90, 91, 92, 93, 94, 95, 96, 97, 98, or 99%) of said CD3−CD56+ cells express at least one molecule selected from CD161 and activating receptors selected from the group comprising or consisting of NKp30, NKp44, NKp46, DNAM-1 and NKG2D.

In one embodiment, the NK cells susceptible to be obtained, or obtained, by the method of the invention express CD161, NKp30, NKp44, NKp46, DNAM1 and NKG2D. In one embodiment, the NK cells susceptible to be obtained, or obtained, by the method of the invention are or comprise CD3−CD56+ cells, wherein at least 80%, preferably at least 85% (e.g., at least 80, 81, 82, 83, 84, 85, 86, 87, 88, 89, 90, 91, 92, 93, 94, 95, 96, 97, 98, or 99%) of said CD3−CD56+ cells express CD161, NKp30, NKp44, NKp46, DNAM-1 and NKG2D.

In one embodiment, the NK cells susceptible to be obtained, or obtained, by the method of the invention express at least one molecule selected from CD161, CD62L and activating receptors selected from the group comprising or consisting of NKp30, NKp44, NKp46, DNAM1 and NKG2D. In one embodiment, the NK cells susceptible to be obtained, or obtained, by the method of the invention express CD161, NKp30, NKp44, NKp46, DNAM1, NKG2D and CD62L.

In one embodiment, at least about 80, 81, 82, 83, 84, 85, 86, 87, 88, 89, 90, 91, 92, 93, 94, 95, 96, 97, 98 or 99% of the NK cells susceptible to be obtained, or obtained, by the method of the invention express at least one molecule selected from CD161 and activating receptors selected from the group comprising or consisting of NKp30, NKp44, NKp46, DNAM1 and NKG2D. In one embodiment, at least about 80, 81, 82, 83, 84, 85, 86, 87, 88, 89, 90, 91, 92, 93, 94, 95, 96, 97, 98 or 99% of the NK cells susceptible to be obtained, or obtained, by the method of the invention express CD161, NKp30, NKp44, NKp46, DNAM1 and NKG2D.

In one embodiment, at least about 50, 55, 60, 65 or 75% of the NK cells susceptible to be obtained, or obtained, by the method of the invention express at least one molecule selected from CD161 and activating receptors selected from the group comprising or consisting of NKp30, NKp44, NKp46, DNAM1 and NKG2D. In one embodiment, at least about 50, 55, 60, 65 or 75% of the NK cells susceptible to be obtained, or obtained, by the method of the invention express CD161, NKp30, NKp44, NKp46, DNAM1 and NKG2D.

In one embodiment, the NK cells susceptible to be obtained, or obtained, by the method of the invention express CD62L. In one embodiment, at least about 1, 2, 3, 4, 5, 6, 7, 8, 9, 10, 11, 12, 13, 14, 15, 16, 17, 18, 19, 20, 21, 22, 23, 24 or 25% of the NK cells express CD62L.

In one embodiment, the NK cells susceptible to be obtained, or obtained, by the method of the invention express CCR5. In one embodiment, at least about 70, 71, 72, 73, 74, 75, 76, 77, 78, 79, 80, 85, 90, 95% of the NK cells express CCR5.

In one embodiment, the NK cells susceptible to be obtained, or obtained, by the method of the invention do not express at least one of inhibitory receptor selected from the group comprising or consisting of KIR2DL1/2/3, KIR3DL1, KIR3DL2, CD94/NKG2A, LIR-1, KLRG-1, CEACAM1, TIGIT, Siglec-3, -7, -9, LAIR-1 and CD300A.

In one embodiment, the NK cells susceptible to be obtained, or obtained, by the method of the invention do not express at least one inhibitory receptor selected from the group comprising or consisting of KIR2DL1/2/3, KIR3DL1, KIR3DL2, and KLRG-1. In one embodiment, the NK cells susceptible to be obtained, or obtained, by the method of the invention do not express KIR2DL1/2/3, KIR3DL1, KIR3DL2, nor KLRG-1.

In one embodiment, less than about 1, 2, 3, 4, 5, 6, 7, 8, 9, 10, 11, 12, 13, 14 or 15% of the NK cells susceptible to be obtained, or obtained, by the method of the invention express at least one inhibitory receptor selected from the group comprising or consisting of KIR2DL1/2/3, KIR3DL1, KIR3DL2, and KLRG-1.

In one embodiment, less than about 1, 2, 3, 4, 5, 6, 7, 8, 9, 10, 11, 12, 13, 14 or 15% of the NK cells susceptible to be obtained, or obtained, by the method of the invention express CD16.

In one embodiment, less than about 15, 16, 17, 18, 19, 20, 21, 22, 23, 24, 26, 27, 28, 29, 30, 31, 32, 33, 34; 35, 36, 37, 38, 39, 40, 41, 42, 43, 44, 45, 46, 47, 48, 49, 50, 51, 52, 53, 54 or 55% of the NK cells susceptible to be obtained, or obtained, by the method of the invention express CD16.

In one embodiment, at least about 35, 40 or 45% of the NK cells susceptible to be obtained, or obtained, by the method of the invention express CD94 and/or NKG2A. In one embodiment, at least about 15, 20, 25 or 30% of the NK cells susceptible to be obtained, or obtained, by the method of the invention express CD94 and/or NKG2A. In one embodiment, at least about 50, 55, 60, 65, 70 or 75% of the NK cells susceptible to be obtained, or obtained, by the method of the invention express CD94 and/or NKG2A.

In one embodiment, the NK cells susceptible to be obtained, or obtained by the method of the invention are (or comprise) immature NK cells. Immature NK cells may for example lack expression of KIR receptors and/or of CD16.

In one embodiment, the NK cells susceptible to be obtained, or obtained, by the method of the invention are mature NK cells. Mature NK cells may for example express CD16 and/or KIR receptors.

In one embodiment, the NK cells susceptible to be obtained, or obtained, by the method of the invention are (or comprise) mature NK cells, such as, for example, memory-like NK cells.

In one embodiment, the NK cells susceptible to be obtained, or obtained, by the method of the invention comprise immature NK cells and mature NK cells (e.g., memory-like NK cells).

The present invention further relates to a NK cell population, wherein NK cells express CD161, DNAM-1 NKp30, NKp44, NKp46 and NKG2D and do not express KIR3DL1/DL2, KIR3DL2/DL3, KLRG1. In one embodiment, said NK cell population is isolated.

The present invention further relates to a NK cell population, wherein NK cells are or comprise CD3−CD56+ cells, wherein at least 80%, preferably at least 85% (e.g., at least 80, 81, 82, 83, 84, 85, 86, 87, 88, 89, 90, 91, 92, 93, 94, 95, 96, 97, 98, or 99%) of said CD3−CD56+ cells express at least one molecule selected from CD161 and activating receptors selected from the group comprising or consisting of NKp30, NKp44, NKp46, DNAM-1 and NKG2D.

In one embodiment, NK cells express CD62L.

The present invention further relates to NK cells expressing CD161, DNAM-1 NKp30, NKp44, NKp46 and NKG2D and not expressing KIR3DL1/DL2, KIR3DL2/DL3, KLRG1. In one embodiment, said NK cells are isolated.

In one embodiment, at least about 80, 81, 82, 83, 84, 85, 86, 87, 88, 89, 90, 91, 92, 93, 94, 95, 96, 97, 98 or 99% of the cells of the NK cell population of the invention express CD161 and the activating receptors NKp30, NKp44, NKp46, DNAM1 and NKG2D. In one embodiment, at least about 50, 55, 60, 65 or 75% of the cells of the NK cell population of the invention express CD161 and the activating receptors NKp30, NKp44, NKp46, DNAM1 and NKG2D.

In one embodiment, the NK cells of the invention express CCR5. In one embodiment, at least about 70, 71, 72, 73, 74, 75, 76, 77, 78, 79, 80, 85, 90, 95% of the NK cells of the population express CCR5.

In one embodiment, at most about 1, 2, 3, 4, 5, 6, 7, 8, 9, 10, 11, 12, 13, 14 or 15%, more preferably at most about 2% of the NK cells of the population express the inhibitory receptors KIR2DL1/2/3, KIR3DL1, KIR3DL2 and KLRG-1.

In one embodiment, at most about 1, 2, 3, 4, 5, 6, 7, 8, 9, 10, 11, 12, 13, 14 or 15% of the NK cells of the population of the invention express CD16.

In one embodiment, less than about 1, 2, 3, 4, 5, 6, 7, 8, 9, 10, 11, 12, 13, 14 or 15% of the NK cells of the population of the invention express CD16.

In one embodiment, less than about 15, 16, 17, 18, 19, 20, 21, 22, 23, 24, 26, 27, 28, 29, 30, 31, 32, 33, 34; 35, 36, 37, 38, 39 or 40% of the NK cells of the population of the invention express CD16.

In one embodiment, less than about 41, 42, 43, 44, 45, 46, 47, 48, 49, 50, 51, 52, 53, 54 or 55% of the NK cells of the population of the invention express CD16.

In one embodiment, at least about 35, 40 or 45% of the NK cells of the population express CD94. In one embodiment, at least about 15, 20, 25 or 30% of the NK cells of the population of the invention express CD94.

In one embodiment, at least about 35, 40 or 45% of the NK cells of the population express NKG2A. In one embodiment, at least about 15, 20, 25 or 30% of the NK cells of the population of the invention express NKG2A. In one embodiment, at least about 50, 55, 60, 65, 70 and 75% of the NK cells of the of the population of the invention express NKG2A.

In one embodiment, less than about 1, 2, 3, 4, 5, 6, 7, 8, 9, 10, 11, 12, 13, 14 or 15% of the NK cells of the population of the invention express NKG2A.

In one embodiment, at least about 15, 20, 25, 30, 35, 40 or 45% of the NK cells of the population express NKG2A and CD94.

In one embodiment, the NK cells of the invention are immature NK cells. In one embodiment, the NK cell population of the invention comprises immature NK cells. In one embodiment, the NK cells of the invention are mature NK cells (e.g., memory-like NK cells)/In one embodiment, the NK cell population of the invention comprises mature NK cells (e.g., memory-like NK cells). In one embodiment, the NK cell population of the invention comprises immature NK cells and mature NK cells (e.g., memory-like NK cells).

In one embodiment, the NK cells of the present invention (that may for example be obtained by the method of the present invention) are functional, i.e., are capable of cytotoxic activity.

The function of NK cells may be verified by conventional methods known in the art. Examples of such methods include, without limitation, cytotoxicity assays, measurement of the secretion of IFNγ or of TNF-α and measurement of the production (and capacity of degranulation) of perforin and granzyme. Examples of such methods are presented in the experimental part.

In one embodiment, the NK cells are cytotoxic in vitro in the condition of TEST A.

In practice, TEST A is a flow cytometry-based cytotoxicity assay, carried out as follows: target cells (e.g., K562 cells or THP1 cells) are labeled with CellTrace Violet dye by incubating them with 1 μM of the dye at 37° C. for 10 min, to distinguish the target cells from effector NK cells. The labeled target cells are then incubated with NK cells at different effector to target ratios in RPMI medium supplemented with 10% FBS and 30 IU/ml of hIL-2 for 5 hrs at 37° C. inside CO₂ incubator. After 5 hrs of incubation, the cells are stained with 7-AAD to distinguish the target cells killed by the NK cells. The effect of spontaneous target cell death is normalized (by substracting the % spontaneous target cell death without effector cells from the % target cell deaths in presence of effector cells) by including one condition of incubation of only target cells without NK cells. In one embodiment, in TEST A, a positive control condition corresponds to a condition wherein target cells are exposed to a detergent, such as, for example, Tween 20, diluted at about 0.2% in PBS, thereby measuring total target cell death.

In one embodiment, the cells are cytotoxic in the conditions of TEST A if the percentage of dead cells among target cells is of at least about 10% in at least one effector to target cell ratio, and the following criteria are met: percentage of spontaneous target cell death is of inferior or equal to about 5%, percentage of total target cell death is superior or equal to about 99%; and a coefficient of variation between replicates is inferior or equal to about 20%.

In one embodiment, the cells are cytotoxic in the conditions of TEST A if at a ratio effector: target of 1.25:1, a percentage of cytotoxicity of at least about 5, 10 or 15% is measured, along with a dose dependent response, meaning that the more the ratio effector: target cells is important, the more the cytotoxicity of NK cells is high.

In one embodiment, the cells are cytotoxic in the conditions of TEST A if at a ratio effector: target of 1.25:1, a percentage of cytotoxicity of at least about 20% is measured, along with a dose dependent response, meaning that the more the ratio effector: target cells is important, the more the cytotoxicity of NK cells is high.

In one embodiment, the NK cells of the present invention express a CAR. In one embodiment, the NK cells of the present invention express a protein selected from the group comprising or consisting of cytokines or cytokines receptors or variants thereof (such as, for example, variants of cytokines or cytokines receptors with increased stability). In one embodiment, the NK cells of the present invention express a chimeric cytokine receptor or orthogonal cytokine-receptor pairs. In one embodiment, the NK cells of the present invention express IL-15 or a variant thereof. In one embodiment, the NK cells of the present invention express CD16 or a variant thereof (such as, for example, cleavage resistant variant of CD16). In one embodiment, the NK cells of the present invention do not express at least one gene selected from the group comprising or consisting of PD1, TIGIT, LAG-3, TIM-3, Cytokine induced STAT inhibitor (CIS) and Signal regulatory ρrotein a (SIRPα). In one embodiment, the NK cells of the present invention do not express at least one gene selected from the group comprising or consisting of PD1, TIGIT, LAG-3, TIM-3, TGFB2, Cytokine induced STAT inhibitor (CIS) and Signal regulatory protein α (SIRPα).

The present invention further relates to a kit for performing the method of the invention comprising: TNF-α or a fragment thereof, a Notch ligand or fragment thereof, and at least three (such as, for example, 3, 4 or 5, and preferably five) cytokines selected from the group comprising or consisting of interleukin-7 (preferably hIL-7), Stem Cell Factor (preferably hSCF), Interleukin-15 (preferably hIL-15), Interleukin-2 (preferably hIL-2) and Flt3 ligand (preferably hFLT3L). In one embodiment, the kit for performing the method of the invention may comprise IL-7 (preferably hIL-7), SCF (preferably hSCF), IL-15 (preferably hIL-15) and FLT3L (preferably hFLT3L). In one embodiment, the kit further comprise fibronectin or a fragment thereof. In one embodiment, the kit further comprises IL-12 (preferably hIL-12) and/or IL-18 (preferably hIL-18).

Such kit is a particularly adapted and designed for performing the method herein disclosed.

Another object of the present invention is a NK cell population as described herein, for increasing or for use in increasing the number of NK cells in a subject in need thereof.

Another object of the present invention is a NK cell population as described herein for use in therapy, such as, for example, in “off-the-shelf” therapy.

Another object of the present invention is a NK cell population as described herein for use as a medicament.

The present invention further relates to the use of a NK cell population as described herein, for the manufacture of a medicament for increasing the number of NK cells in a subject.

The present invention further relates to a method for increasing the number of NK cells in a subject in need thereof, comprising administering to the subject a NK cell population as described herein (in particular a therapeutically effective amount of NK cells as described herein).

The present invention further relates to a composition comprising, consisting essentially of or consisting of a NK cell population according to the present invention.

In one embodiment, the composition is a pharmaceutical composition and further comprises at least one pharmaceutically acceptable excipient. Consequently, the present invention further relates to a pharmaceutical composition.

In one embodiment, the pharmaceutical composition comprises, consists essentially of or consists of a NK cell population according to the present invention and at least one pharmaceutically acceptable excipient.

Pharmaceutically acceptable excipients that may be used in the pharmaceutical composition of the invention include, but are not limited to, ion exchangers, alumina, aluminum stearate, lecithin, serum proteins, such as, for example, human serum albumin, buffer substances such as, for example, phosphates, glycine, sorbic acid, potassium sorbate, partial glyceride mixtures of saturated vegetable fatty acids, water, salts or electrolytes, such as, for example, protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene-block polymers, polyethylene glycol and wool fat.

In one embodiment, the composition of the present invention is, or is for use as, a medicament. Consequently, the present invention further relates to a medicament.

In one embodiment, the medicament comprises, comprises, consists essentially of or consists of a NK cell population according to the present invention.

As used herein, the term “consisting essentially of”, with reference to a composition, pharmaceutical composition or medicament, means that the NK cells of the invention are the only one therapeutic agents or agents with a biologic activity within said composition, pharmaceutical composition or medicament.

In one embodiment, the NK cell population, composition, pharmaceutical composition or medicament of the invention is for treating cancer or an infectious disease.

The present invention thus relates to NK cells, a NK cell population, a composition, pharmaceutical composition or medicament as disclosed herein for treating cancer or an infectious disease.

The present invention further relates to the use of NK cells or of a NK cell population as disclosed herein for the manufacture of a medicament for treating cancer or an infectious disease.

The present invention further relates to a method for treating cancer or an infectious disease in a subject in need thereof, comprising administering to the subject a NK cell population as described herein (in particular a therapeutically effective amount of NK cells as described herein).

In one embodiment, the subject is a human.

In one embodiment, the subject is affected, preferably diagnosed with a cancer.

Examples of cancers include, but are not limited to leukemia (i.e., acute myeloid leukemia), lymphoma (e.g., B lymphomas), non-Hodgkin lymphoma, multiple myeloma, breast cancer, bladder cancer, prostate cancer, pancreatic cancer, thyroid cancer, melanoma, uterine cancer, kidney cancer, sarcoma, carcinoma, non-small cell lung cancer, oral and oropharyngeal cancer, methylcholanthrene-induced sarcomas and colorectal cancer.

Examples of cancers include, but are not limited to leukemia (e.g., acute myeloid leukemia, B cell acute lymphoblastic leukemia (B-ALL), T cell acute lymphoblastic leukemia (T-ALL), chronic lymphocytic leukemia (CLL), chronic myeloid leukemia), lymphoma (e.g., B lymphomas, peripheral T cell lymphoma), non-Hodgkin lymphoma, glioblastoma, neuroblastoma, multiple myeloma, cervical cancer, breast cancer (e.g., Triple-negative breast cancer), ovarian cancer, bladder cancer, prostate cancer, pancreatic cancer, gastric cancer, thyroid cancer, melanoma, uterine cancer, kidney cancer, liver cancer (e.g., hepatocellular cancer), sarcoma, carcinoma (e.g., renal cell carcinoma, breast carcinoma), small cell lung cancer, non-small cell lung cancer, pediatric solid tumor, CD133+ cancer stem cells, NKGDL+ cancer cells, PD-L1+ cancer cells, oral and oropharyngeal cancer (e.g., tongue cancer, esophageal cancer, laryngeal cancer, pharyngeal cancer) methylcholanthrene-induced sarcomas and colorectal cancer.

In one embodiment, the subject is affected, preferably is diagnosed, with an infectious disease.

In one embodiment, the subject is affected, preferably is diagnosed, with a viral persistent infection caused by a virus selected from the group comprising or consisting of human immunodeficiency virus (HIV), herpesvirus (e.g., herpes simplex virus-1, cytomegalovirus (CMV)) influenza, retroviruses, human papillomavirus (HPV), enteroviruses (e.g., coxsackie B3 virus).

In one embodiment, the subject is affected, preferably is diagnosed, with a parasitic infection. Examples of parasitic infections include, but are not limited to, toxoplasmosis, trypanosomiasis, leishmaniasis and malaria.

In one embodiment of the invention, the subject to be treated is administrated at least once with the therapeutically effective amount of the composition as described here above.

In one embodiment, a single dose of NK cells of the present invention is administered to the subject. In another embodiment, a plurality of doses of NK cells of the present invention are administered over a period of time.

In one embodiment, a therapeutically effective amount of NK cells is administered, is for administration or is to be administered to the subject to be treated. In one embodiment, the therapeutically effective amount ranges from about 0.5×10⁷ to about 3×10⁷ cells/kg body weight.

In one embodiment, the number of NK cells that may be injected to a subject ranges from about 0.5×10⁷ to about 3×10⁷ cells/kg body weight.

In one embodiment, the NK cells, the NK cell population, the composition, the pharmaceutical composition or the medicament of the invention is administered (or is to be administered or is for administration) by intravesical administration, intravaginal administration, intraosseous administration, intraperitoneal administration, intrauterine administration, intraocular administration, intradermal administration, intraarterial administration, intracerebral administration, intranasal administration, enteral administration, buccal administration, intranasal administration, oral administration, rectal administration, or by inhalation.

In one embodiment, the NK cells, the NK cell population, the composition, the pharmaceutical composition or the medicament of the invention is administered (or is to be administered or is for administration) by injection, including, without limitation, subcutaneous, intravenous, intramuscular, intra-articular, intra-synovial, intra-sternal, intrathecal, intrahepatic, intralesional and intracranial injection or infusion techniques.

Examples of forms adapted for injection include, but are not limited to, solutions, such as, for example, sterile aqueous solutions, gels, dispersions, emulsions, suspensions, solid forms suitable for using to prepare solutions or suspensions upon the addition of a liquid prior to use, such as, for example, powder, liposomal forms and the like.

### BRIEF DESCRIPTION OF THE DRAWINGS

FIG. 1 is a combination of dot plots and histograms showing the generation of CD3−CD56+ NK cells from CB or mPB CD34+ HSPCs using DL-4/TNF-α culture system. (A) FIG. 1A is a combination of dot plots representative of the phenotype of NK cell generated after 7-days of culture in a DL-4/TNF-α medium followed by a DL-4/TNF-α-free NK cell differentiation medium at the indicated time points. (B, C) FIG. 1B and FIG. 1C are a combination of graphs showing the mean frequencies (B) and numbers per CD34+ cell (C) of CD3−CD56+ NK cells at day 14 (light grey) and at day 21 (dark grey) (mean±SEM, n=5).

FIG. 2 is a combination of a histogram and a graph showing the expression of NK cell receptors and transcription factors by the CB or mPB HSPCS (DL-4/TNF-α-exposed)-derived NK cells (CD3−CD56+). (A, B) FIG. 2A and FIG. 2B are graphical representation of the expression percentages of NK cell receptors (mean±SEM, n=2 or 3) (A) and of transcription factors (Eomes, T-bet and ID-2) (B) by the CD3−CD56+ NK cells generated after 21 days of culture. The expression levels were compared to those of control overnight activated peripheral blood (PB)-NK cells (in presence of hIL-2 and IL15).

FIG. 3 is a combination of dot plots showing the expression of cytotoxic molecules by CB or mPB NK cells (CD3−CD56+) and their cytokine expression after stimulation with K562 cells. (A) FIG. 3A is a combination of representative dot plots showing the expression of cytotoxic granules (perforin and granzyme B) by the generated CD3−CD56+ NK cells. (B, C) FIG. 3B and FIG. 3C are combinations of representative dot plots showing the degranulation (expression of CD107a on cell surface) (B), and expression of TNF-α and IFNg (C) by CD3−CD56+ NK cells upon 6 hrs of stimulation with K562 cells at Effector (NK): Target (K562) ratio of 1:2.

FIG. 4 is a combination of dot plots and histograms showing the generation of transduced NK cells from CB or mPB CD34+ HSPCSs using DL-4/TNF-α culture system. (A) FIG. 4A is a combination of representative dot plots showing the generation of transduced (GFP+CD3−CD56+) NK cells (B, C) FIG. 4B and FIG. 4C are histograms showing the mean frequencies (B) and numbers per CD34+ cell (C) of transduced GFP+CD3−CD56+ NK cells generated at day 14 (light grey) and at day 21 (dark grey) after transduced NK cell generation cultures (mean±SEM, n=5).

FIG. 5 is a histogram showing the expression of NK cells receptors (mean±SEM, n=2 or 3) by the transduced GFP+CD3−CD56+ NK cells generated.

FIG. 6 is a combination of dot plots showing degranulation and cytokine expression of transduced CB or mPB NK cells after stimulation with K562 cells. FIGS. 6A and B are combinations of representative dot plots showing the degranulation (expression of CD107a on cell surface) (A) induction of TNF-α and IFNg expression (B) and secretion of cytotoxic granules (granzyme B and perforin).

FIG. 7 is a combination of graphs showing the cytotoxicity of the in vitro generated transduced NK cells. (A) FIG. 7A is a graphical representation of the killing of K562 cells (target cells) by transduced CB or mPB NK cells (effector cells) after their co-incubation at the indicated Effector: Target cell (E: T) ratios for 5 hrs (one representative experiment). (B) FIG. 7B is a graphical representation of the killing of THP1 cells (target cells) by transduced CB NK cells (effector cells) after their co-incubation at the indicated Effector: Target cell (E: T) ratios for 5 hrs (one representative experiment).

FIG. 8 is a graph showing the expression of GATA3 and BCL11B by CD7+ cells obtained after culturing CD34+ cells in the presence of TNFα and of a notch ligand, according to the first step of the method of the present invention.

FIG. 9 is a combination of graphs showing the generation of functionally potent CAR NK cells from CB CD34+ HSPCs using DL-4/TNFα culture system. (A) FIG. 9A is a representative FACS plot showing the generation of CAR (CAR⁺CD56⁺) NK cells after NK cell generation cultures that includes transduction with CD19-CAR encoding lentiviruses during the first step of 7-day DL-4/TNFα cultures and a second step of feeder cell-free and DL-4/TNFα-free NK cell differentiation for 8 days. (B) FIG. 9B is a graphical representation of the killing of target cells (NALM-6) by CD19-CAR NK cells (effector cells) after their co-incubation at the indicated Effector: Target cell (E:T) ratios for 5 hrs.

### EXAMPLES

The present invention is further illustrated by the following examples.

### Example 1: Functional CD3−CD56+ NK Cells are Generated

### Material and Methods

### CD34+ Cells

The umbilical cord blood (CB) samples were collected via ethically approved procedures from donors at Saint Louis Hospital (Paris, France) following the provision of informed consent. The mobilized peripheral blood (mPB) samples used were the unused fraction of grafts from healthy donors mobilized with granulocyte colony-stimulating factor and who had provided their informed consent for research use. The mPB samples were part of a collection authorized by the French Ministry of Research (reference: DC-2014-2272, dated Mar. 23, 2015). Cord blood or mPB CD34+hematopoietic stem and progenitor cells (HSPCSs) were magnetically enriched from CB and mPB samples using the Indirect CD34 MicroBead Kit, human (Miltenyi Biotech, Bergisch Gladbach, Germany) according to the manufacturers' instruction. The purity of the isolated CD34+ cell fraction used were >94%.

### In Vitro NK Cell Differentiation Assay

The in vitro production of NK cells was performed in two steps in a total of 21 days:

The 1st step consisted of 7-day culture of human CD34+ cells in DL-4/TNF-α culture system. The CB or mPB CD34+ HSPCs were cultured on DL-4-Fc fusion protein (5 μg/ml) and RetroNectin® (25 μg/ml)-coated wells in α-MEM medium (Thermo Fischer, MA, USA) supplemented with 20% FBS (Hyclone, GE Healthcare Life Sciences), 100 ng/ml of hSCF, 100 ng/ml of hTPO, 100 ng/ml of hFlt3-L and 100 ng/ml of hIL-7 in the presence of TNF-α (10 ng/ml) for 7 days.

The 2nd step consisted of 14-day culture of the progenitors (total population without any sorting of a particular cell population) obtained from the 1st step in a feeder cell-free culture system with a human cytokine cocktail but without DL-4 and TNF-α to generate NK cells. The progenitors from the 1st step were cultured in non-coated wells (No DL-4) for 14 days in RPMI Glutamax medium (Thermo Fischer, MA, USA) supplemented with 10% FBS (Hyclone, GE Healthcare Life Sciences), 50 ng/ml of hSCF, 50 ng/ml of hFlt3-L, 20 ng/ml of hIL-7, 20 ng/ml of hIL-15 (Peprotech), 500 IU/ml of hIL-2 (Novartis) and in the absence of TNF-α for 14 days to obtain the NK cell product.

### Flow Cytometry

Antibodies against human CD56-APCVio770/PEVio770 (Clone REA196), CD161-PEVio770 (Clone 191B8), NKG2C (CD159c)-APC (Clone REA205), KLRG1-PE (Clone REA261), CD158e/k (KIR3DL1/DL2)-PEVio770 (Clone REA970), CD158b (KIR2DL2/DL3)-APC (Clone DX27), NKG2D (CD314)-APC (Clone REA797), NKG2A (CD159a)-PEVio770 (Clone REA110), CD94-PE (Clone REA113), NKp44 (CD336)-PEVio770 (Clone REA1163), NKp30 (CD337)-APC (Clone REA823), CD16-Vioblue (Clone REA423), DNAM-1 (CD226)-PEVio770 (Clone REA1040), T-bet-APC (Clone REA102), CD107a-PEVio770 (Clone REA792), TNF-α-PE (Clone REA656), IFNg-APC (Clone 45-15), Perforin-PE (Clone REA1061), Granzyme B-APC (Clone REA226) and 7-Aminoactinomycin D (7-AAD) were obtained from Miltenyi Biotech (Bergisch Gladbach, Germany). Anti-human CD7-PE (clone MT701) and NKp46-PE (Clone 9E2/NKp46 (9-E2) were from BD Biosciences (San Jose, CA). Anti-human-CD3-BV421 (UCHT1) was purchased from Sony Biotechnology (San Jose, CA). Antibodies against human CCR5 (CD195)-APC (Clone J418FI) was from Biolegend (San Diego, CA). Anti-human Eomes-PE (Clone WD1928) and ID2-PECy7 (Clone ILCID2) were from eBioscience (San Diego, CA).

For surface staining, the cells were incubated with the appropriate antibodies for 15 min in ice, washed and then resuspended in FACS buffer.

For intracellular staining, the cells were pre-stained for surface markers, fixed and permeabilized using either Fixation/Permeabilization Solution Kit (BD Biosciences) or Foxp3/Transcription Factor Staining Buffer Set (eBioscience) according to the manufacturers' instructions and then incubated with the appropriate antibodies for 30 min at room temperature. The cells were then washed and resuspended in FACS buffer before analysis.

All flow cytometry data were acquired in Gallios flow cytometer (Beckman Coulter, Krefeld, Germany) and the data were analyzed using FlowJo software (version 10.2, Treestar, Ashland, OR). During the FACS analyses, all the gatings were done on live cells (determined by the exclusion of 7-AAD dye).

### Degranulation and Cytokine Induction Assay

The CB or mPB CD34+ HSPCS-derived NK cells (CB HSPCS-NK or mPB HSPCS-NK) obtained were stimulated with K562 cells (chronic myelogenous leukemia cell line) by incubating together in a 1:2 Effector (E) to target (T) ratio at 37° C. inside CO2 incubator without any cytokines. Anti-human CD107a antibody was added (20 ul/ml) at the time of incubation and then incubated for 1 hr. After 1 hr, Brefeldin (GolgiStop) (4 ul/6 ml) and Monensin (GolgiPlug) (lul/ml) were added and further incubated for 5 hrs. After 5 hrs of incubation, the cells were collected, washed and surface stained for CD3 and CD56. They were further stained intracellularly for TNF-α and IFNg. The stained cells were acquired in Gallios flow cytometer.

### Cytotoxicity Assays

Flow cytometry-based cytotoxicity assay was performed by using K562 and cells as target cells. The target cells (K562 or THP1) were labeled with CellTrace Violet dye by incubating them with 1 uM of the dye at 37° C. for 10 min, to distinguish the target cells from effector NK cells. The labeled target cells were then incubated with effector NK cells at different effector to target ratios in RPMI medium supplemented with 10% FBS and 30 IU/ml of hIL-2 for 5 hrs at 37° C. inside CO2 incubator. After 5 hrs of incubation, the cells were stained with 7-AAD to distinguish the target cells killed by the effector NK cells. The effect of spontaneous target cell death was normalized (by substracting the % spontaneous target cell death without effector cells from the % target cell deaths in presence of effector cells) by including one condition of incubation of only target cells without effector cells. Peripheral blood derived NK cells were used as positive control effector cells in parallel to the in vitro generated NK cells.

For all the experiments, two controls were used. CB CD34+ cells treated only with the second step medium (CB CD34+, no DL-4/TNF-α) and NK from peripheral blood activated overnight with IL-2 and IL-15 (PB-NK).

### Results

### Generation of NK CD3−CD56+ Cells

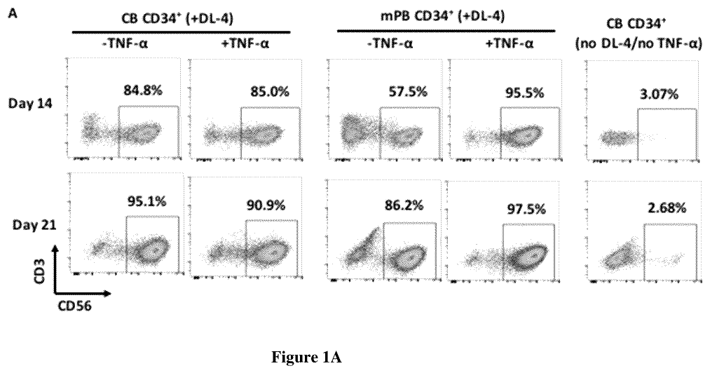

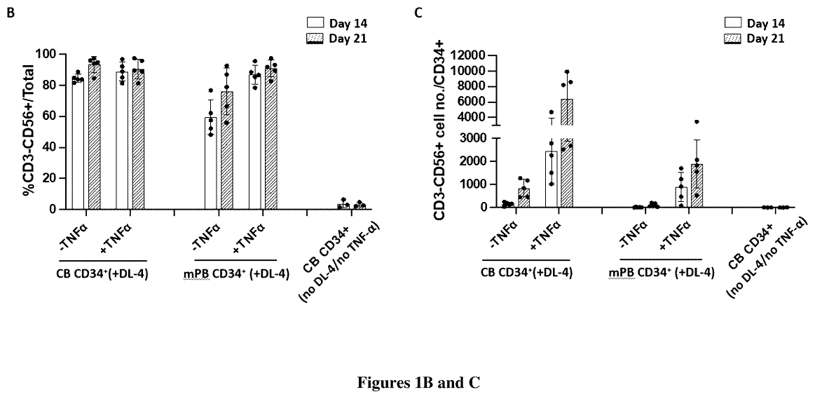

To test the potential of NK cell generation using DL-4/TNF-α cultures from CB or mPB CD34+ HSPCs, the CD34+ cells were cultured on DL-4 and Retronectin-coated wells for 7 days in presence or absence of TNF-α (10 ng/ml) followed by a DL-4/TNF-α-free NK cell differentiation cultures of the cells obtained after the initial 7 days of culture (CB CD34+ (+DL-4/+TNF-α) or mPB CD34+ (+DL-4/+TNF-α)). In parallel, CB CD34+ HSPCs, not exposed to DL-4/TNF a (CB CD34+ (no DL-4/no TNF-α)) were also cultured with a DL-4/TNF-α-free NK cell differentiation medium. The cultures were analyzed after 14 and 21 days of total culture for their differentiation into NK cells (identified as CD3−CD56+ cells). The NK cell (CD3−CD56+ cell) differentiation is very low (up to 3%) for CB CD34+ (no DL-4/no TNF-α), whereas the DL-4 exposed HSPCs, CB CD34+ (+DL-4) or mPB CD34+ (+DL-4), were able to efficiently differentiate into CD3−CD56+NK cells without any T cell contamination for both TNF-α-exposed or non-exposed conditions within a short period of 14 and 21 days. Interestingly, the percentage of NK cells reached up to 90% since day 14 in the presence of TNF-α (FIGS. 1, A and B). The purity of the NK cells generated was more homogeneous in TNF-α-exposed condition especially for adult mPB cells (mPB CD34+ (+DL-4)) (FIG. 1B). Importantly, the analysis of the total NK cell yield showed that the addition of TNF-α during the initial 7-day DL-4 exposure of CB or mPB CD34+ HSPCs, highly increases the number of NK cells obtained per CD34+ HSPCS by 8- and 18-fold respectively for CB and mPB after 21 days of culture (FIG. 1C). However, the number of NK cells obtained from no-DL-4/no TNF-α-exposed CB CD34+ cells remained strikingly low as shown in FIG. 1C. Moreover, analysis of the NK cell production using+DL-4/+TNF-α cultures from different CD34+ HSPCs donors showed that a single CB CD34+ HSPCs could give rise to an average of 2488±1832 (mean±SEM) NK cells for CB and 879±805 (mean±SEM) NK cells for mPB at day 14 and an average of 6389±3723 (mean±SEM) NK cells for CB and 1881±1458 (mean±SEM) NK cells for mPB at day 21 under TNF-α-exposed conditions (FIG. 1C). These data demonstrate that the combination of the 7-day first step of culture in the presence of +DL-4/+TNF-α and the second step of culture in a DL-4/TNF-α-free medium can generate a high percentage and number of NK cells during a short culture period of 14 to 21 days.

### Characterization of the Generated NK Cells

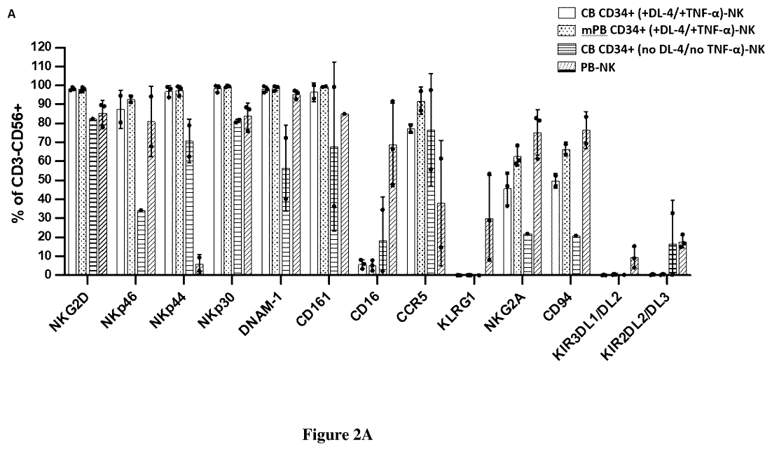

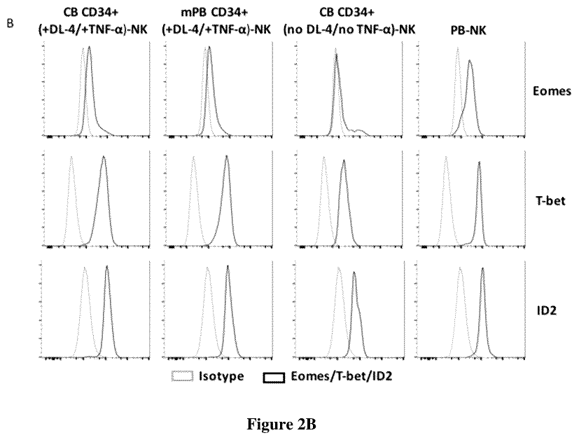

The phenotypic characterization of the CB or mPB CD34+ HSPCs (+DL-4/+TNF-α-exposed)-derived NK cells generated, showed that the NK cells express the activating receptors NKG2D, NKp46, NKp44, NKp30, DNAM-1 and express CD161, but do not express the inhibitory receptors KLRG1, KIR2DL2/DL3 and KIR3DL1/DL2 (FIG. 2A and Table 1). Importantly, they also expressed the chemokine receptor CCR5 even at higher levels than those of activated PB-NK cells. However, only a low percentage of these cells (ranging from about 5 to about 8%) showed CD16 expression and about 50% of them express NKG2A (expressing percentage ranging from about 45% to about 63%) and CD94 (expressing percentage ranging from about 50% to about 66%) (FIG. 2A and Table 1). The CD34+ (no DL-4/no TNF-α-exposed) derived-NKs expressed lower levels of expression of the activating receptors as compared to those of CB or mPB NK cells (+DL-4/+TNF-α). The activated PB-NK cells showed some level of expression of KLRG1, which is an exhaustion marker for NK cell. Thus, the CB or mPB NK cells generated were phenotypically distinct from activated PB-NK cells in terms of the expression of CD16, KIRs and KLRG1 which were either expressed at very low levels or not expressed in the CB or mPB NK cells (+DL-4/+TNF-α) (FIG. 2A and Table 1). Notably, while the NKp44 was poorly expressed by activated PB-NK cells, the CB or mPB NK cells (+DL-4/+TNF-α) highly expressed NKp44 (FIG. 2A and Table 1). The lack of expression of KIRs and KLRG1, and very low expression of CD16 (FIG. 2A and Table 1), in combination with the homogeneous expression of CD56 (FIG. 1A), suggested an immature phenotype of the CB or mPB NK cells (+DL-4/+TNF-α). Interestingly, similar to the activated PB-NK cells, the CB or mPB NK (+DL-4/+TNF-α) cells also express the transcription factors Eomes, T-bet and ID2 (FIG. 2B) which are known to be essential for NK cell differentiation and function.

TABLE 1
Comparison of CB-NK cells, mPB-NK cells and PB-NK phenotype
(mean ± SEM; n = 2 or n = 3)

| | CB-HSPC NK | mPB HSPC-NK | PB-NK |
|---|---|---|---|
| Activation | | | |
| Receptors | | | |
| NKG2D | 98.2 ± 0.6% | 98.0 ± 0.8% | 85.5 ± 5.1% |
| NKp46 | 87.5 ± 10.6% | 92.7 ± 1.4% | 81.1 ± 1.0% |
| NKp44 | 97.0 ± 1.9% | 97.4 ± 2.0% | 5.9 ± 3.5% |
| NKp30 | 98.4 ± 1.4% | 99.5 ± 0.4% | 83.9 ± 5.3% |
| DNAM-1 | 97.9 ± 1.2% | 98.6 ± 1.1% | 95.3 ± 1.7% |
| CD16 | 5.8 ± 1.6% | 5.2 ± 1.8% | 68.8 ± 15.4% |
| Inhibitory | | | |
| Receptors | | | |
| KLRG1 | 0.1 ± 0.1% | 0.1 ± 0.0% | 30.1 ± 15.4% |
| NKG2A | 45.8 ± 6.2% | 62.6 ± 3.9% | 75.1 ± 9.3% |
| CD94 | 49.7 ± 2.7% | 66.4 ± 2.6% | 76.4 ± 7.0% |
| KIR3DL1/DL2 | 0.2 ± 0.2% | 0.5 ± 0.2% | 9.4 ± 3.9% |
| KIR2DL2/DL3 | 0.4 ± 0.3% | 0.4 ± 0.1% | 18.0 ± 3.5% |
| Chemokine | | | |
| Receptor | | | |
| CCR5 | 77.3 ± 1.7% | 91.8 ± 5.1% | 38.5 ± 23.5% |
| Other | | | |
| CD161 | 99.0 ± 0.9% | 99.6 ± 0.2% | 85.1 ± 2.0% |

### The NK Cells Generated are Functional

### Degranulation and Perforin and Granzyme Expression

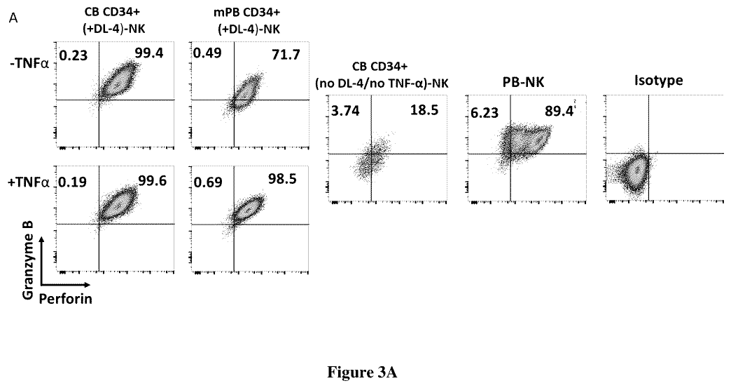

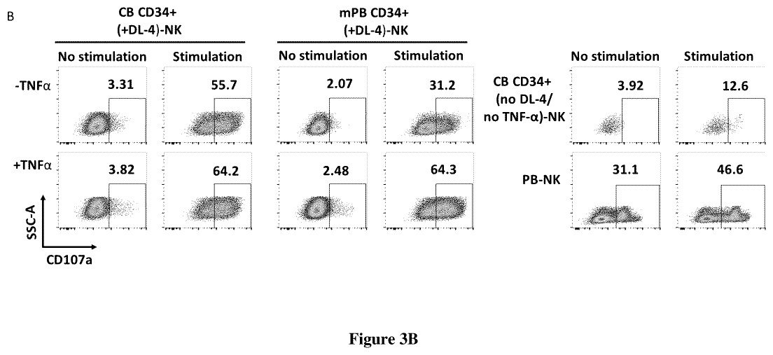

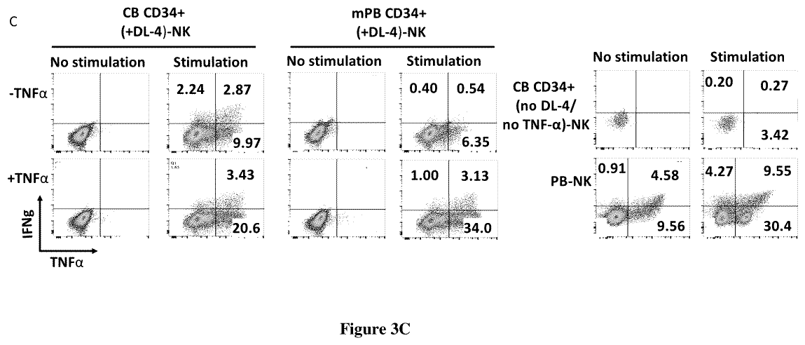

To determine the functionality of the CB or mPB NK cells generated, first they were analyzed for the expression of the cytotoxic molecules perforin and granzyme B, which are known to be constitutively expressed by NK cells and are important for inducing target cell killing. Similar to the activated PB-NK cells, the CB or mPB NK cells (both for non-TNF-α exposed or TNF-α-exposed) expressed both perforin and granzyme B (FIG. 3A), reflecting their ability to be cytotoxic against their target cells. However, the CB CD34+-NK (no DL-4/no TNF-α) cells expressed lower levels of perforin and granzyme B than CB or mPB NK (+DL-4/with or without TNF-α) and PB-NK cells (FIG. 3A).

Next, the NK cells were assessed for their ability to undergo degranulation (which is an important process to secrete the cytotoxic molecules to kill target cells) and to induce Interferon gamma (IFNg) and TNF-α (that can mediate target cell killing by inducing apoptosis) expression upon stimulation with their target cells. Upon stimulation with myelogenous leukemia cell line K562 cells, the CB or mPB NK (+DL-4/with or without TNF-α) cells showed degranulation as indicated by the detection of CD107 on their cell surface and were comparable to those of activated PB-NK cells (FIG. 3B). The level of degranulation was higher in TNF-α-exposed condition as compared to their non-exposed counterpart, particularly for mPB. In addition to the lower perforin and granzyme B expression (as mentioned above), the degranulation level was also lower in CB CD34+-NK cells (no DL-4/no TNF-α) as compared to CB or mPB NK cells (+DL-4/with or without TNF-α) (FIG. 3B). IFNg and TNF-α were induced upon stimulation with K562 cells under all conditions except for CB CD34+-NK cells (no DL-4/no TNF-α) (FIG. 3C). However, importantly, the CB or mPB NK cells derived from TNF-α-exposed conditions showed higher TNF-α induction than their non-TNFα-exposed counterparts. Moreover, specifically for mPB, the IFNg induction was lower in non-TNF-α-exposed conditions (FIG. 3C). Taken together, these data suggest that the NK cells generated by the method of the invention are functional.

### Cytotoxicity

The cytotoxic activity of the CB or mPB NK cells (+DL-4/+TNF-α) was tested by incubating the NK cells with K562 cells, used as target cells. The CB and mPB NK cells (+DL-4/+TNF-α) NK cells were able to efficiently kill K562 cells (data not shown). These data suggests that the NK cells generated with the method of the invention possess cytotoxic potential and can kill their target cells in vitro.

### Example 2: CD34+ Cells can be Efficiently Transduced and Differentiate into CD3-CD56+ NK Cells

### Material and Methods

### Transduction Protocol

During the first step of 7-day culture, the CB or mPB CD34+ HSPCs were preactivated overnight on DL-4-Fc fusion protein (5 μg/ml) and RetroNectin® (25 μg/ml)-coated wells at a cell concentration of 1×10⁶ cells/ml in X-vivo 20 medium (Lonza) in the presence of human (h) cytokines-300 ng/ml hSCF, 100 ng/ml hTPO, 300 ng/ml hFlt3-L, 100 ng/ml hIL-7, 20 ng/ml hIL-3 (Peprotech) and in absence of TNF-α (R and D Systems). The preactivated cells were then transduced for 6 hrs in the same preactivation medium in presence of 4 μg/ml of Protamine Sulfate with VSV-G pseudotyped lentiviruses encoding a GFP reporter protein at a multiplicity of infection (MOI) of 100. After 6 hrs of transduction, the transduced cells were washed with α-MEM medium (Thermo Fischer, MA, USA) and the transduction media were replaced by α-MEM medium supplemented with 20% FBS (Hyclone, GE Healthcare Life Sciences), 100 ng/ml of hSCF, 100 ng/ml of hTPO, 100 ng/ml of hFlt3-L and 100 ng/ml of hIL-7 in the presence of TNF-α (10 ng/ml) and further cultured on DL-4 and RetroNectin coated wells until 7 days.

The 2nd step consisted of 14-day culture of the progenitors (total population without any sorting of a particular cell population) obtained from the 1st step in a feeder cell-free culture system with a human cytokine cocktail but without DL-4 and TNF-α to generate NK cells. The 1st step progenitors were cultured in non-coated wells (No DL-4) for 14 days in RPMI Glutamax medium (Thermo Fischer, MA, USA) supplemented with 10% FBS (Hyclone, GE Healthcare Life Sciences), 50 ng/ml of hSCF, 50 ng/ml of hFlt3-L, 20 ng/ml of hIL-7, 20 ng/ml of hIL-15 (Peprotech), 500 IU/ml of hIL-2 (Novartis) and in the absence of TNF-α for 14 days to obtain the NK cell product.

### Results

### Generation of Transduced NK CD3−CD56+ Cells

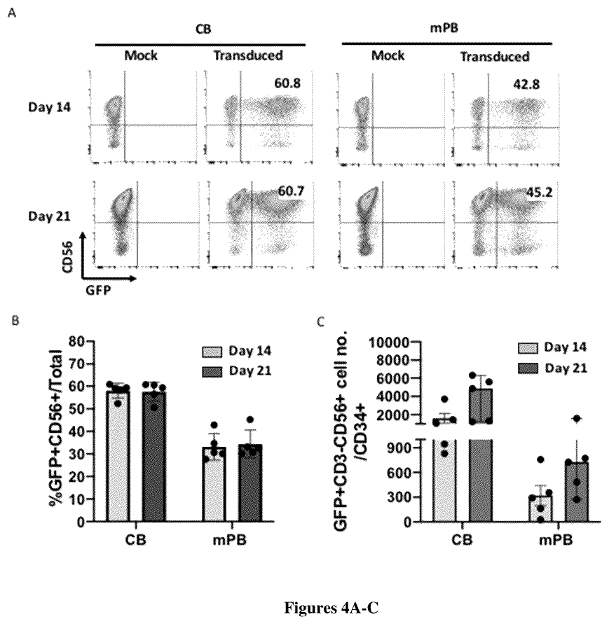

To test whether transduced NK cells could be generated by using the present method, the CB or mPB CD34+ cells were preactivated overnight on DL-4 and Retronectin-coated wells in a transduction cytokine cocktail and then transduced for 6 hrs with a GFP-expressing VSV-G pseudotyped lentivirues. After transduction, the transduced cells were further cultured until 7 days in presence of TNF-α (10 ng/ml) in DL-4 and Retronectin-coated wells in a culture cytokine cocktail. This was followed by a DL-4/TNF-α-free NK cell differentiation culture of the cells obtained after the initial 7 days of culture. The cultures were analyzed after 14 and 21 days of cultures for their differentiation into transduced NK cells (identified as CD3-GFP+CD56+ cells). As shown in FIGS. 4, A and B, transduced GFP+CD3−CD56+ NK cells were observed within a short culture period of 14 and 21 days with their mean frequencies at 58±4.5 (mean±SEM) for CB and 33±7.5 (mean±SEM) for mPB at day 21. The analysis of the total transduced GFP+CD3−CD56+ NK cell yield showed that a single CB CD34+ HSPCS could give rise to an average of 1600±1442 (mean±SEM) transduced NK cells for CB and 318±365 (mean±SEM) transduced NK cells for mPB at day 14 and an average of 3857±2537 (mean±SEM) transduced NK cells for CB and 770±664 (mean±SEM) transduced NK cells for mPB at day 21 (FIG. 4C). These data demonstrate that the present culture methods can generate a high numbers of transduced NK cells during a short culture period of 14 to 21 days.

### Characterization of the Generated NK Cells

Similar to the untransduced NK cells generated by the method of the invention (FIG. 2A), transduced GFP+CD3−CD56+CB or mPB NK cells (+DL-4/+TNF-α) express the activation receptors NKG2D, NKp46, NKp44, NKp30, DNAM-1 and express CD161, but do not express the inhibitory receptors KLRG1, KIR2DL2/DL3 and KIR3DL1/DL2 (FIG. 5). Importantly, they also express the chemokine receptor CCR5. They express very low CD16, and NKG2A and CD94 were expressed by only 50% of the cells (FIG. 5). This data suggest that the transduction condition had no impact on the expression of NK receptors in transduced NK cells.

### The NK Cells Generated are Functional

### Degranulation and Perforin and Granzyme Expression

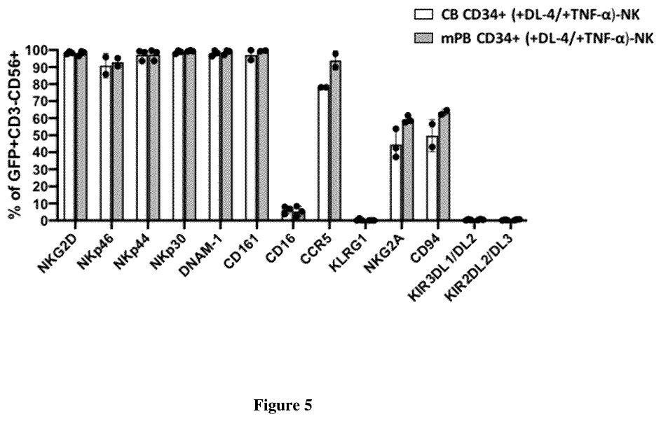

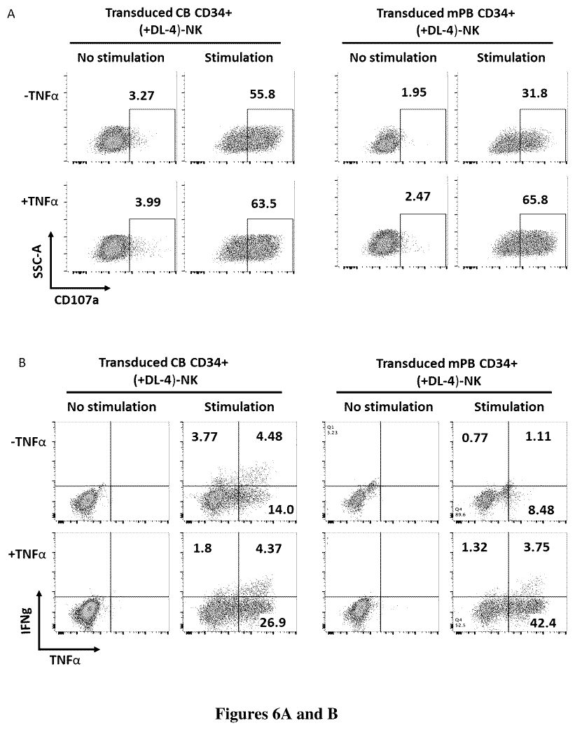

To assess the functional potential of the transduced NK cells generated, degranulation and cytokine induction assay was performed by stimulating the transduced CB or mPB NK cells (+DL-4/with or without TNF-α) with K562 cells. Upon stimulation, the transduced CB or mPB NK cells (+DL-4/with or without TNF-α) showed degranulation as indicated by the detection of CD107 on their cell surface. (FIG. 6A). The level of degranulation was higher in TNF-α-exposed condition as compared to their non-exposed counterpart, especially for mPB (FIG. 6A). IFNg and TNF-α expression were also induced in the transduced CB or mPB NK cells (+DL-4/with or without TNF-α) upon stimulation with K562 cells (FIG. 6B). Interestingly, the transduced CB or mPB NK cells derived from TNF-α-exposed conditions showed higher TNF-α induction than their non-TNF-α-exposed counterparts. Moreover, the IFNg induction was lower in non-TNF-α-exposed conditions especially for mPB (FIG. 6B). Finally, granzyme B and perforin expression were also induced in the transduced CB or mPB NK cells (+DL-4/with TNF-α) upon stimulation with K562 cells (data not shown). These data suggest that the transduced CB or mPB NK cells still retain their functional capacity.

### Cytotoxicity

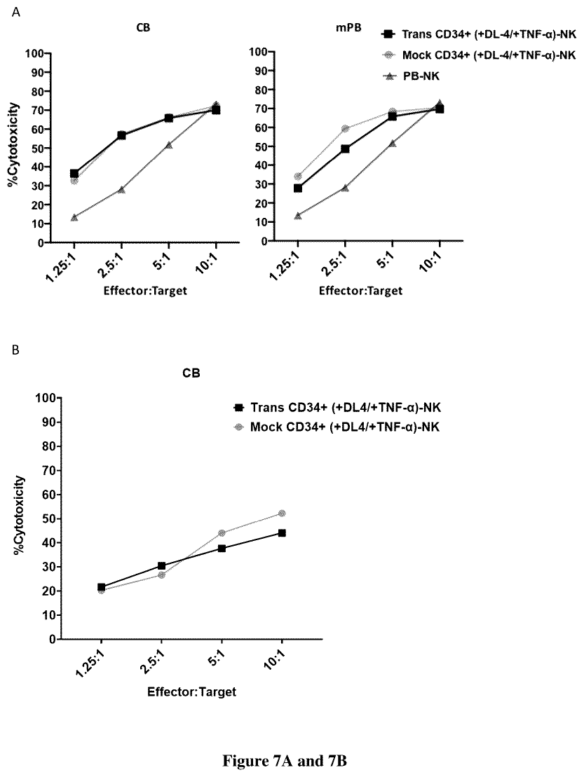

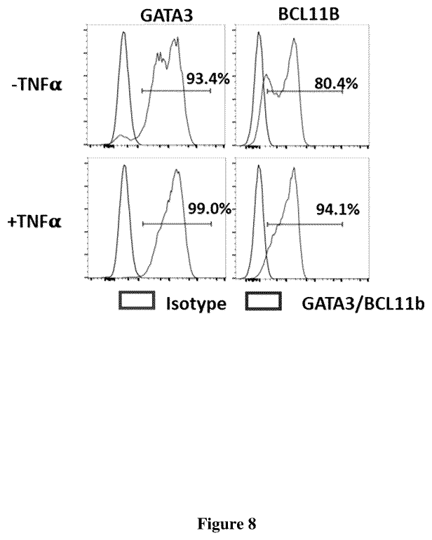

The cytotoxic activity of the transduced CB or mPB NK cells (+DL-4/+TNF-α) were tested by incubating the NK cells with K562 cells or THP1 cells as target cells. As shown in FIG. 7A, the transduced CB or mPB NK cells (+DL-4/+TNF-α), obtained by the method of the invention, were able to efficiently kill K562 cells at similar levels to those of untransduced (Mock) conditions or PB-NK, suggesting that the transduction had no impact on the cytotoxic potential of the transduced NK cells. Moreover, the transduced CB NK cells (+DL-4/+TNF-α), obtained by the method of the invention, were also able to efficiently kill THP1 cells (FIG. 7B). These data suggest that the transduced NK cells generated with the method of the invention can efficiently kill their target cells in vitro.

### Example 3: Production of Functionally Potent CAR NK Cells from CB CD34+ HSPCs

### Material and Methods

### Transduction Protocol

During the first step of 7-day culture, the CB CD34+ HSPCs were preactivated overnight on DL-4-Fc fusion protein (5 μg/ml) and RetroNectin® (25 μg/ml)-coated wells at a cell concentration of 1×10⁶ cells/ml in X-vivo 20 medium (Lonza) in the presence of human (h) cytokines: 300 ng/ml hSCF, 100 ng/ml hTPO, 300 ng/ml hFlt3-L and 100 ng/ml hIL-7 (Peprotech) and in absence of TNFα (R&D Systems). The preactivated cells were then transduced for 6 hours in the same preactivation medium in presence of 4 μg/ml of Protamine Sulfate with VSV-G pseudotyped lentiviruses encoding a CAR targeting CD19 at a multiplicity of infection (MOI) of 100. After 6 hours of transduction, the transduced cells were washed with α-MEM medium (Gibco) and the transduction media were replaced by α-MEM medium supplemented with 20% FBS (Hyclone, GE Healthcare Life Sciences), 100 ng/ml of hSCF, 100 ng/ml of hTPO, 100 ng/ml of hFlt3-L and 100 ng/ml of hIL-7 in the presence of TNFα (10 ng/ml) and further cultured on DL-4 and RetroNectin coated wells until 7 days.

The second step consisted of 8-day culture of the progenitors (total population without any sorting of a particular cell population) obtained from the first step in a feeder cell-free culture system with a human cytokine cocktail but without DL-4 and TNFα to generate NK cells. The progenitors obtained after the first step of culture were cultured in non-coated wells (No DL-4) for 8 days in RPMI Glutamax medium (Gibco) supplemented with 10% FBS (Hyclone, GE Healthcare Life Sciences), 50 ng/ml of hSCF, 50 ng/ml of hFlt3-L, 20 ng/ml of hIL-7, 20 ng/ml of hIL-15 (Peprotech), 500 IU/ml of hIL-2 (Novartis) and in the absence of TNFα to obtain the NK cell product.

### Cytotoxicity Assays

Flow cytometry-based cytotoxicity assay was performed by using NALM-6 (B cell precursor leukemia cell line) cells as target cells. The NALM-6 target cells were labeled with CellTrace Violet dye by incubating them with 1 μM of the dye at 37° C. for 10 min, to distinguish the target cells from effector NK cells. The labeled target cells were then incubated with effector NK cells at different effector to target ratios in RPMI medium supplemented with 10% FBS and 30 IU/ml of hIL-2 for 5 hours at 37° C. inside a CO₂ incubator. After 5 hours of incubation, the cells were stained with 7-AAD to distinguish the target cells killed by the effector NK cells. The effect of spontaneous target cell death was normalized (by substracting the % spontaneous target cell death without effector cells from the % target cell deaths in presence of effector cells) by including one condition of incubation of only target cells without effector cells.

### Results

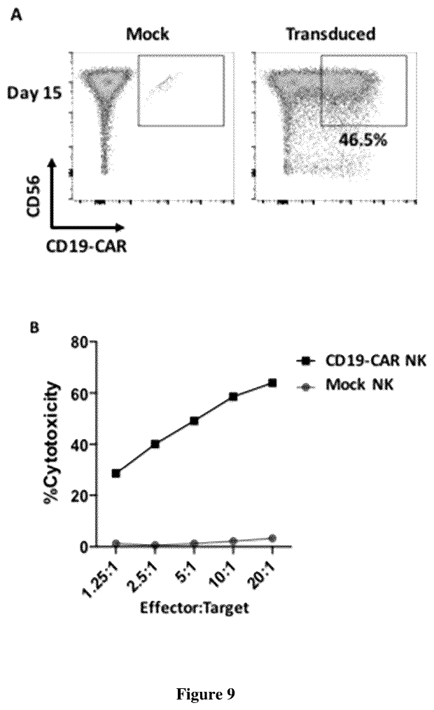

To test whether chimeric antigen receptor (CAR) expressing NK cells could be generated by using the present method, the CB CD34+ cells were preactivated overnight on DL-4 and Retronectin-coated wells in a transduction cytokine cocktail and then transduced for 6 hours with VSV-G pseudotyped lentiviruses encoding CAR targeting against CD19. After transduction, the transduced cells were further cultured until 7 days in presence of TNFα (10 ng/ml) in DL-4 and Retronectin-coated wells in DL-4 culture cytokine cocktail. This was followed by a feeder cell and DL-4/TNFα-free NK cell differentiation cultures of the progenitors obtained after the initial 7 days of culture. The cultures were analyzed after 8 days for their differentiation into CAR expressing NK cells (identified as CD3-CAR+CD56+ cells). As shown in FIG. 9A, CAR+CD56+ NK cells were observed within a short culture period of 15 days with a frequency of 46.5%. These data demonstrate that the present culture methods can generate CAR NK cells during a short culture period of 15 days.

The cytotoxic activity of the CD19 targeting CAR NK cells generated were tested by incubating the NK cells with NALM-6 (B cell precursor leukemia cell line which express CD19) cells as target cells. As shown in FIG. 9B, the CD19-CAR NK cells were able to efficiently kill NALM-6 cells in contrast to the mock NK cells which were not able to kill the target NALM-6 cells. These data suggest that the CAR NK cells generated with the current method could efficiently kill their specific target cells in vitro.

### Example 4: Phenotypic Characterization of CD3−CD56+ NK Cells and CAR-NK Cells Generated with or without IL-12 and IL18

### Material and Methods

### In Vitro NK Cell Differentiation Assay

The in vitro production of NK cells was performed in two steps in a total of 14 days:

The 1st step consisted of 7-day culture of human CD34+ cells in DL-4/TNF-α culture system. The CB CD34+ HSPCs were cultured on DL-4-Fc fusion protein (5 μg/ml) and RetroNectin® (25 μg/ml)-coated wells in α-MEM medium (Thermo Fischer, MA, USA) supplemented with 20% FBS (Hyclone, GE Healthcare Life Sciences), 100 ng/ml of hSCF, 100 ng/ml of hTPO, 100 ng/ml of hFlt3-L and 100 ng/ml of hIL-7 in the presence of TNF-α (10 ng/ml) for 7 days.

The 2nd step consisted of 14-day culture of the progenitors (total population without any sorting of a particular cell population) obtained from the 1st step in a feeder cell-free culture system with a human cytokine cocktail but without DL-4 and TNF-α to generate NK cells. The progenitors from the 1st step were cultured in non-coated wells (No DL-4) for 14 days in RPMI Glutamax medium (Thermo Fischer, MA, USA) supplemented with 10% FBS (Hyclone, GE Healthcare Life Sciences), 50 ng/ml of hSCF, 50 ng/ml of hFlt3-L, 20 ng/ml of hIL-7, 20 ng/ml of hIL-15 (Peprotech), 500 IU/ml of hIL-2 (Novartis), with or without 10 ng/ml of hIL-12 (Peprotech), 100 ng/ml of hIL-18 (MBL International Corporation) and in the absence of TNF-α for 7 days to obtain the NK cell product.

### Flow Cytometry

Antibodies against human CD56-APCVio770/PEVio770 (Clone REA196), NKG2C (CD159c)-APC (Clone REA205), KLRG1-PE (Clone REA261), CD158e/k (KIR3DL1/DL2)-PEVio770 (Clone REA970), CD158b (KIR2DL2/DL3)-APC (Clone DX27), NKG2D (CD314)-APC (Clone REA797), NKG2A (CD159a)-PEVio770 (Clone REA110), CD94-PE (Clone REA113), NKp44 (CD336)-PEVio770 (Clone REA1163), DNAM-1 (CD226)-PEVio770 (Clone REA1040) and 7-Aminoactinomycin D (7-AAD) were obtained from Miltenyi Biotech (Bergisch Gladbach, Germany). Anti-human CD7-PE (clone MT701), NKp46-PE (Clone 9E2/NKp46 (9-E2) and CD62L-BV421 (Clone M-T701), were from BD Biosciences (San Jose, CA). Anti-human-CD3-BV421/BV510 (UCHT1) was purchased from Sony Biotechnology (San Jose, CA). Anti-human NKp30 (CD337)-BV421 (Clone DREG-56) and CD16-BV510 (3G8) were obtained from Biolegend (San Diego, CA).

For surface staining, the cells were incubated with the appropriate antibodies for 15 min in ice, washed and then resuspended in FACS buffer.

All flow cytometry data were acquired in Gallios flow cytometer (Beckman Coulter, Krefeld, Germany) and the data were analyzed using FlowJo software (version 10.2, Treestar, Ashland, OR). During the FACS analyses, all the gatings were done on live cells (determined by the exclusion of 7-AAD dye).

### Transduction Protocol

During the first step of 7-day culture, the CB CD34+ HSPCs were preactivated overnight on DL-4-Fc fusion protein (5 μg/ml) and RetroNectin® (25 μg/ml)-coated wells at a cell concentration of 1×10⁶ cells/ml in X-vivo 20 medium (Lonza) in the presence of human (h) cytokines: 300 ng/ml hSCF, 100 ng/ml hTPO, 300 ng/ml hFlt3-L and 100 ng/ml hIL-7 (Peprotech) and in absence of TNFα (R&D Systems). The preactivated cells were then transduced for 6 hours in the same preactivation medium in presence of 4 μg/ml of Protamine Sulfate with VSV-G pseudotyped lentiviruses encoding a ZsG reporter protein at a multiplicity of infection (MOI) of 100. After 6 hours of transduction, the transduced cells were washed with α-MEM medium (Gibco) and the transduction media were replaced by α-MEM medium supplemented with 20% FBS (Hyclone, GE Healthcare Life Sciences), 100 ng/ml of hSCF, 100 ng/ml of hTPO, 100 ng/ml of hFlt3-L and 100 ng/ml of hIL-7 in the presence of TNFα (10 ng/ml) and further cultured on DL-4 and RetroNectin coated wells until 7 days.

The second step consisted of 8-day culture of the progenitors (total population without any sorting of a particular cell population) obtained from the first step in a feeder cell-free culture system with a human cytokine cocktail but without DL-4 and TNFα to generate NK cells. The progenitors obtained after the first step of culture were cultured in non-coated wells (No DL-4) for 8 days in RPMI Glutamax medium (Gibco) supplemented with 10% FBS (Hyclone, GE Healthcare Life Sciences), 50 ng/ml of hSCF, 50 ng/ml of hFlt3-L, 20 ng/ml of hIL-7, 20 ng/ml of hIL-15 (Peprotech), 500 IU/ml of hIL-2 (Novartis), with or without 10 ng/ml of hIL-12, 100 ng/ml of hIL-18 and in the absence of TNFα to obtain the NK cell product.

### Result

The phenotypic characterization of the CD34+ HSPCs (+DL-4/+TNF-α-exposed)-derived NK cells and CD34+ HSPCs (+DL-4/+TNF-α-exposed)-derived CAR-NK cells generated with or without IL-12 and IL18, showed that the NK cells and CAR NK cells express the activating receptors NKG2D, NKp46, NKp44, NKp30, DNAM-1 and CD62L, but do not express the inhibitory receptors KLRG1, KIR2DL2/DL3 and KIR3DL1/DL2 (Table 2). NK cells and CAR NK cells express also the inhibitory receptors CD96 and NKG2A. Moreover, NK cells and CAR NK cells generated in presence of hIL-12 and hIL18 express higher percentage of CD62L (respectively 16 and 19% against 4%) and CD16 (respectively 50 and 44% against 22 and 15%) activating receptors and higher percentage of CD94 (respectively 26 and 30% against 16 and 13%) and NKG2A (respectively 24 and 28% against 11 and 10%) inhibiting receptors compare to NK cells and CAR NK cells generated without hIL-12 and hIL-18. (Table 2)

TABLE 2
Comparison of NK cells and CAR-NK cells phenotype culturing with or without
hIL-12 and hIL18 at Day 14. (mean ± SEM; n = 3)

| | NK cells | | Transduced NK cells | |
|---|---|---|---|---|
| Activation | | | | |
| Receptors | | | | |
| | With hIL-12 | Without hIL-12 | With hIL-12 | Without hIL-12 |
| | and hIL-18 | and hIL-18 | and hIL-18 | and h IL-18 |
| NKG2D | 84.8 ± 13.5% | 89.4 ± 6.8% | 97.3 ± 0.2% | 95.5 ± 0.5% |
| NKp46 | 79.7 ± 2.8% | 84.3 ± 4.4% | 71.6 ± 0.7% | 78.2 ± 3.8% |
| NKp44 | 90.9 ± 1.6% | 68.7 ± 5.3% | 90.8 ± 1.1% | 64.3 ± 2.4% |
| NKp30 | 84.1 ± 12.4% | 95.6 ± 4.4% | 79.7 ± 13.9% | 87.4 ± 7% |
| DNAM-1 | 99.5 ± 0.3% | 98.4 ± 1% | 99.4 ± 0.4% | 98 ± 1.1% |
| CD16 | 43.1 ± 5.1% | 8.6 ± 6.6% | 35.6 ± 6.8% | 9.4 ± 2.9% |
| Inhibitory | | | | |
| Receptors | | | | |
| | With hIL-12 | Without hIL-12 | With hIL-12 | Without hIL-12 |
| | and hIL-18 | and hIL-18 | and hIL-18 | and hIL-18 |
| KLRG1 | 2.8 ± 2.3% | 0.2 ± 0.2% | 0.4 ± 0.1% | 0.2 ± 0.4% |
| NKG2A | 33.4 ± 5.3% | 5.9 ± 2.7% | 34.3 ± 4% | 5.9 ± 1.8% |
| CD94 | 32.2 ± 5% | 7.2 ± 4.2% | 30.1 ± 8% | 32.7 ± 9.8% |
| KIR3DL1/DL2 | 0.1 ± 0.1% | 0 ± 0.01% | 0.2 ± 0.1% | 0.3 ± 0.1% |
| KIR2DL2/DL3 | 1.3 ± 0.9% | 0.5 ± 0.3% | 1.7 ± 1.1% | 0.5 ± 0.3% |
| Adhesion | | | | |
| molecules | | | | |
| | With hIL-12 | Without hIL-12 | With hIL-12 | Without hIL-12 |
| | and hIL-18 | and hIL-18 | and hIL-18 | and h IL-18 |
| CD62L | 39.6 ± 11.6% | 8 ± 3% | 49 ± 14.9% | 13.1 ± 5.6% |
| Others | | | | |
| CD161 | 70.55 ± 16.9% | 99.2 ± 0.2% | 78 ± 8.8% | 99.1 ± 14.3% |

1-15. (canceled)

16. An in vitro method for generating NK cells, comprising the steps of:
a) culturing CD34+ cells in presence of TNF-α or of a fragment thereof and of a Notch ligand or fragment thereof, thereby obtaining a first population of cells, and
b) culturing the population of cells obtained in step a) in a cytokine comprising medium.

17. The in vitro method according to claim 16, wherein at step a), the cells are cultured in presence of TNF-α or of a fragment thereof and of a Notch ligand or fragment thereof for more than 5 days and less than 9 days.

18. The in vitro method according to claim 16, wherein at step a), the cells are cultured in presence of TNF-α or of a fragment thereof and of a Notch ligand or fragment thereof for about 7 days.

19. The in vitro method according to claim 16, wherein the Notch ligand is the Delta-like-4 ligand or a fragment thereof.

20. The in vitro method according to claim 19, wherein the Notch ligand is the soluble domain of the Delta-like-4 ligand.

21. The in vitro method according to claim 16, wherein at step a), the cells are also exposed to a fibronectin fragment comprising the RGDS, connecting segment 1 (CS-1) and/or heparin-binding domain.

22. The in vitro method according to claim 21, wherein the fibronectin fragment is CH-296.

23. The in vitro method according to claim 16, wherein said CD34+ cells have been isolated from an adult donor or from cord blood cells.

24. The in vitro method according to claim 16, wherein the cytokine comprising medium of step (b) contains at least three cytokines, selected from the group consisting of interleukin-7 (IL-7), Stem Cell Factor (SCF), Interleukin-15 (IL-15), Interleukin-2 (IL-2) and Flt3 ligand (FLT3L).

25. The in vitro method according to claim 16, wherein the cytokine comprising medium of step (b) contains interleukin-7 (IL-7), Stem Cell Factor (SCF), Interleukin-15 (IL-15), Interleukin-2 (IL-2) and Flt3 ligand (FLT3L).

26. The in vitro method according to claim 16, wherein, at step b), the cells are cultured in the cytokine comprising medium for more than 7 days and less than 21 days.

27. The in vitro method according to claim 16, comprising an additional step of transducing cells with a vector.

28. The in vitro method according to claim 27, wherein the additional step of transducing cells with a vector is carried out during or before step (a).

29. The in vitro method according to claim 27, wherein the vector encodes a Chimeric Antigen Receptor (CAR).

30. A NK cell population susceptible to be obtained by the in vitro method according to claim 16, wherein more than 60% of the cells are CD3−CD56+.

31. The NK cell population according to claim 30, wherein the cells are CD3−CD56+ cells and do not express at least one inhibitory receptor selected from the group comprising KIR3DL1/DL2, KIR2DL2/DL3 and KLRG1.

32. The NK cell population according to claim 30, wherein the cells are CD3−CD56+ cells and express at least one molecule selected from CD161 and activating receptors selected from the group comprising NKp30, NKp44, NKp46, DNAM-1 and NKG2D.

33. A NK cell population expressing CD161, NKp30, NKp44, NKp46, DNAM-1 and NKG2D and not expressing KIR3DL1/DL2, KIR3DL2/DL3, KLRG1.

34. A method for treating cancer or an infectious disease in a subject in need thereof comprising administering to the subject a NK cell population according to claim 30.

35. A method for treating cancer or an infectious disease in a subject in need thereof comprising administering to the subject a NK cell population according to claim 33.


SEQUENCE LISTING (9 sequences)

SEQ ID NO: 1 - protein, 233 aa, synthetic construct
  REGION 1..233: note = TNF-alpha
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - protein, 685 aa, synthetic construct
  REGION 1..685: note = Delta-like-4
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 3 - protein, 223 aa, synthetic construct
  REGION 1..223: note = IgG2Fc
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 4 - protein, 754 aa, synthetic construct
  REGION 1..754: note = Delta-like-4-IgG2Fc
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSTMVRSVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 2477 aa, synthetic construct
  REGION 1..2477: note = fibronectin
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 6 - protein, 4 aa, synthetic construct
  REGION 1..4: note = RGDS motif
RGDS

SEQ ID NO: 7-8 - skipped (2 entries)

SEQ ID NO: 9 - protein, 25 aa, synthetic construct
  REGION 1..25: note = CS-1 motif
DELPQLVTLPHPNLHGPEILDVPST